(19) United States
(12) Patent Application Publication
(10) Pub. No.: US 2020/0093181 A1
(43) Pub. Date: Mar. 26, 2020

(54) FLAVORANTS

(71) Applicant: RAI Strategic Holdings, Inc., Winston-Salem, NC (US)

(72) Inventors: Sawyer Austin Hubbard, Winston-Salem, NC (US); Willie Dat Tran, Winston-Salem, NC (US)

(21) Appl. No.: 16/137,047

(22) Filed: Sep. 20, 2018

(51) Int. Cl. A24F 47/00; A24B 15/16; A24D 1/14; A24B 15/40; A61M 11/04; A61M 15/00

(52) U.S. Cl. CPC A24F 47/008; A24B 15/167; A61M 15/009; A24B 15/403; A61M 11/041; A24D 1/14

(57) ABSTRACT

A smoking article is provided herein, which includes a power source, a control component adapted to control power delivery from the power source, a heating element, and an aerosolizable composition comprising at least one flavorant derived from a plant of the humulus lupulus and/or asimina triloba species or at least one flavorant comprising a synthetic compound responsible for the aroma and/or flavor of an artisan food item. The disclosure further provides a method for providing a smoking article with flavors or aromas of craft beer or flavors or aromas of artisan foods.

### FIELD OF THE INVENTION

The present invention relates to flavorants useful for incorporation into products such as tobacco products including, but not limited to, aerosol delivery articles. Of particular interest are flavorants or components obtained or derived from ingredients often associated with the craft beer industry and flavorants or component obtained or synthetically prepared so as to mimic the flavor and/or aroma of artisan or local food items.

### BACKGROUND OF THE INVENTION

Cigarettes, cigars, and pipes are popular smoking articles that employ tobacco in various forms. Such smoking articles are employed by heating or burning tobacco to generate aerosol (e.g., smoke) that may be inhaled by the smoker. Many smoking devices have been proposed through the years as improvements upon, or alternatives to, smoking products that require combusting tobacco for use. Many of those devices purportedly have been designed to provide the sensations associated with cigarette, cigar, or pipe smoking, but without delivering considerable quantities of incomplete combustion and pyrolysis products that result from the burning of tobacco. To this end, there have been proposed numerous smoking products, flavor generators, and medicinal inhalers that utilize electrical energy to vaporize or heat a volatile material, or attempt to provide the sensations of cigarette, cigar, or pipe smoking without burning tobacco to a significant degree. See, for example, the various alternative smoking articles, aerosol delivery devices and heat generating sources set forth in the background art described in U.S. Pat. No. 7,726,320 to Robinson et al., U.S. patent application Ser. No. 13/432,406, filed Mar. 28, 2012, U.S. patent application Ser. No. 13/536,438, filed Jun. 28, 2012, U.S. patent application Ser. No. 13/602,871, filed Sep. 4, 2012, and U.S. patent application Ser. No. 13/647,000, filed Oct. 8, 2012, which are incorporated herein by reference.

Certain tobacco products that have employed electrical energy to produce heat for smoke or aerosol formation, and in particular, certain products that have been referred to as electronic cigarette products, have been commercially available throughout the world. Representative products that resemble many of the attributes of traditional types of cigarettes, cigars or pipes have been marketed as ACCORD® by Philip Morris Incorporated; ALPHA™, JOYE 510™ and M4™ by InnoVapor LLC; CIRRUS™ and FLING™ by White Cloud Cigarettes; COHITA™, COLIBRI™, ELITE CLASSIC™, MAGNUM™, PHANTOM™ and SENSE™ by Epuffer® International Inc.; DUOPRO™, STORM™ and VAPORKING® by Electronic Cigarettes, Inc.; EGAR™ by Egar Australia; eGo-C™ and eGo-T™ by Joyetech; ELUSION™ by Elusion UK Ltd; EONSMOKE® by Eonsmoke LLC; GREEN SMOKE® by Green Smoke Inc. USA; GREENARETTE™ by Greenarette LLC; HALLIGAN™, HENDU™, JET™, MAXXQ™, PINK™ and PITBULL™ by Smoke Stik®; HEATBAR™ by Philip Morris International, Inc.; HYDRO IMPERIAL™ and LXE™ from Crown7; LOGIC™ and THE CUBAN™ by LOGIC Technology; LUCI® by Luciano Smokes Inc.; METRO® by Nicotek, LLC; NJOY® and ONEJOY™ by Sottera, Inc.; NO. 7™ by SS Choice LLC; PREMIUM ELECTRONIC CIGARETTE™ by PremiumEstore LLC; RAPP E-MYSTICK™ by Ruyan America, Inc.; RED DRAGON™ by Red Dragon Products, LLC; RUYAN® by Ruyan Group (Holdings) Ltd.; SMART SMOKER® by The Smart Smoking Electronic Cigarette Company Ltd.; SMOKE ASSIST® by Coastline Products LLC; SMOKING EVERYWHERE® by Smoking Everywhere, Inc.; V2CIGS™ by VMR Products LLC; VAPOR NINE™ by VaporNine LLC; VAPOR4LIFE® by Vapor 4 Life, Inc.; VEPPO™ by E-CigaretteDirect, LLC and VUSE® by R. J. Reynolds Vapor Company. Yet other electrically powered aerosol delivery devices, and in particular those devices that have been characterized as so-called electronic cigarettes, have been marketed under the tradenames BLU™; COOLER VISIONS™; DIRECT E-CIG™; DRAGONFLY™; EMIST™; EVERSMOKE™; GAMUCCI®; HYBRID FLAME™; KNIGHT STICKS™; ROYAL BLUES™; SMOKETIP® and SOUTH BEACH SMOKE™.

It would be desirable to provide a smoking article that employs heat produced by electrical energy to provide the sensations of cigarette, cigar, or pipe smoking, and that incorporates unique flavors in the vapor-producing material(s). It would be further desirable to incorporate such unique flavors in other types of tobacco products.

### BRIEF SUMMARY

The present invention provides tobacco products incorporating ingredients and flavorants often associated with products from the craft brewing industry and/or ingredients and flavorants reminiscent of flavors or aromas often associated with artisan or local foods.

### BRIEF DESCRIPTION OF THE DRAWINGS

In order to provide an understanding of embodiments of the invention, reference is made to the appended drawings, which are not necessarily drawn to scale, and in which reference numerals refer to components of example embodiments of the invention. The drawings are for example only, and should not be construed as limiting the invention.

FIG. 1 is a sectional view through an electronic smoking article comprising a cartridge and a control body and including a reservoir housing according to an example embodiment of the present disclosure;

FIG. 2 schematically illustrates an aerosol delivery device comprising a cartridge body and a control body, the cartridge body being illustrated in an exploded configuration and the control body being illustrated in an assembled configuration according to an example aspect of the present disclosure;

FIG. 3 schematically illustrates the cartridge body of FIG. 2 implementing an additional aerosol generation arrangement, including one or more aerosol-generating elements, according to one aspect of the present disclosure;

FIG. 4 schematically illustrates the additional aerosol generation arrangement of FIG. 3, configured as a cartridge including one or more aerosol-generating elements, according to another aspect of the present disclosure; and

FIG. 5 4 is a cross-sectional view of a smokeless tobacco product embodiment, taken across the width of the product, showing an outer pouch filled with a smokeless tobacco composition and comprising a flavorant as disclosed herein.

### DETAILED DESCRIPTION

The present invention now will be described more fully hereinafter. This invention may, however, be embodied in many different forms and should not be construed as limited to the embodiments set forth herein; rather, these embodiments are provided so that this disclosure will be thorough and complete, and will fully convey the scope of the invention to those skilled in the art. As used in this specification and the claims, the singular forms “a,” “an,” and “the” include plural referents unless the context clearly dictates otherwise. Reference to “dry weight percent” or “dry weight basis” refers to weight on the basis of dry ingredients (i.e., all ingredients except water).

The present disclosure generally relates to products incorporating certain flavorants as will be described herein below. The flavorants are referred to generally herein as plant-derived flavorants or synthetic flavorant compounds and are derived or prepared based on certain specific desired taste and/or aroma characteristics. For example, certain flavorants are derived from a plant of the humulus lupulus. Certain flavorants are derived from (or created to mimic the flavors of) products such as fruits and nuts that are local, artisan, and/or seasonal, such as the fruit of the asimina triloba species.

The types of products encompassed by the present disclosure vary and, as such, in some embodiments, the form of the flavorants can similarly vary, as will be disclosed in detail herein. In certain aspects, the disclosure relates to articles (and the manufacture thereof) that use electrical energy to heat a material (preferably without combusting the material to any significant degree) to form an inhalable substance which include the flavorants. In certain aspects, the disclosure relates to smokeless tobacco products that include the flavorants. In certain aspects, the disclosure relates to so-called “heat-not-burn” products that include the flavorants.

In various embodiments, products and devices are provided which incorporate one or more flavorants traditionally associated with the craft beer industry. Such flavorants may be plant-derived flavorants, for example, derived from a plant of the humulus lupulus species (also referred to as a “hop plant”), e.g., from the flowers of the plant (also referred to herein as “hops”). Hops, often used in beer recipes, thrive largely in two geographic bands that fall roughly between the 35th and 50th parallels on either side of the equator. The vast majority of hops are grown in the United States and Europe, although hops from other countries are also known and can be used herein. A large number of American hops come from Washington, Oregon, and Idaho, but are also grown in many other states. Hops include an alpha acid component that produces the bitterness in beers that many consumers typically associate with the flavor of hops.

Example hop varieties include, but are not limited to, aalst, admiral, agnus, ahtanum, amarillo, aramis, azzacca, bohemie, bor, bramling cross, bravo, brewer's gold, bullion, calypso, cascade, centennial, challenger, chelan, chinook, citra, cluster, coigneau, columbus, comet, crystal, el dorado, ella, eroica, feux-coeur francais, first gold, fuggle, galaxy, galena, glacier, goldings, greenburg, green bullet, hallertau mittlefruh, hallertau herkules, hallertau magnum, hallertau taurus, harmonie, herald, hersbrucker, horizon, huell melon, junga, kohatu, liberty, Lublin, magnum, mandarina bavaria, marynka, merkur, millennium, moteuka, mount hood, mount rainier, mosaic, nelson sauvin, newport, northdown, northern brewer, nugget, opal, pacifica, pacific gem, pacific jade, kazbek, palisade, perle, phoenix, pilgrim, pilot, pioneer, polaris, polnischer lublin, poperinge, premiant, pride of ringwood, progress, rakau, riwaka, rubin, saaz, San Juan ruby red, santiam, saphir, satus, select, simcoe, sladek, smaragd, sonnet golding, sorachi ace, southern cross, spalt, sterling, sticklebract, strisselspalt, styrian atlast, styrian aurora, styrian bobek, styrian celeia, styrian golding, summer, summit, super alpha, super galena, super pride, sybilla, tardif de Bourgogne, target, tettnanger, tillicum, tomyski, topaz, tradition, ultra, vanguard, vic secret, vital, wai-iti, waimea, wakatu, whitbread golding, wilamette, zeus, and zythos. In some embodiments, a single hop variety is employed in the methods and products herein and in some ingredients, a combination of two or more hop varieties are employed. Hops can be selected, e.g., based on the desired aroma characteristics (e.g., flowery, tropical, citrus-like, earthy, grassy, piney, spicy/herbal, etc.). The form of the hop variety or varieties incorporated as a flavorant within products according to the present disclosure can vary. Hops are commercially available, e.g., as whole leaf hops, powders, cones, pellets, and as extracts.

In some embodiments, hops incorporated within products according to the present disclosure are in solid form (e.g., milled, as will be described herein). Hops can be harvested from the plant and directly used (in green form) or can be subjected to various treatment processes such as, refrigeration, freezing, drying (e.g., freeze-drying or spray-drying), irradiation, yellowing, heating, cooking (e.g., roasting, frying or boiling), fermentation, bleaching or otherwise subjected to storage or treatment for later use. Hops are typically physically processed before incorporation into a product as outlined herein. The harvested hops (optionally following any one or more of the treatments referenced above) can be further subdivided into parts or pieces (e.g., shredded, cut, comminuted, pulverized, milled or ground into pieces or parts that can be characterized as filler-type pieces, granules, particulates or fine powders). The harvested hops can be subjected to external forces or pressure (e.g., by being pressed or subjected to roll treatment). The size of the processed material can vary; in preferred embodiments, where solid hop material is used, it is provided in a size suitable for incorporation within the appropriate portion of a smoking article (as will be outlined herein below). For example, in some embodiments, solid hop material is milled so as to be employed in powder form.

In some embodiments, the hops incorporated within products according to the present disclosure are in liquid form. As such, extracts are advantageously used (and can be, in some embodiments, obtained directly from, e.g., whole leaf hops, powders, cones, or pellets by methods known in the art). The material subjected to extraction can be fresh, dried, or freeze-dried, and various solvents can be used (which may affect the composition of the resulting extract). See, for example, U.S. Pat. No. 2,824,803 to Stark, U.S. Pat. No. 3,892,808 to Mitchell, and U.S. Pat. No. 4,282,259 to Wheldon et al., which are incorporated herein by reference in their entireties. Hops typically contain resins, oils, waxes, soluble material such as tannins, proteins, and pectins, and a cellulosic matrix. In certain embodiments, hop extracts are used which include components of the resins and/or oils. In some embodiments, extracts including resin and/or oil components are used. Hop resin generally includes humulones (including alpha acids such as humulone, cohumulone, adhumulone, and posthumulone) and lupulones (including weakly acidic beta acids such as lupulone, colupulone, and adlupulone), and salts thereof. Hop oils include “fixed,” relatively involatile oils and/or volatile, “essential oils” (which are understood to contribute, in large part, to the aroma of hops). According to the present disclosure, in some embodiments, a hop extract is employed which comprises one or more of alpha acids, beta acids, and essential oils.

The composition of a given extract may be determined, at least in part, by the method of extraction employed. According to the present disclosure, hop extracts can be extracted using various solvents and at various temperatures, which may affect the specific components of the hop extract and the amounts of each component in the hop extract. Solvents suitable for hop extraction include, but are not limited to, water and organic solvents (such as methanol, ethanol, acetone, ethyl acetate, tetrahydrofuran, etc.). Extractions can be done at temperatures below room temperature, at room temperature, at elevated temperatures, or under supercritical conditions.

In particular embodiments, an isolated hop oil is employed. Although not limited thereto, in some embodiments, the hop oil is provided by steam distillation. Various isolated hop oils are commercially available and can be used in the products of the present disclosure. Exemplary compounds present in certain hop oils may include, but are not limited to one or more of myrcene, dipentene, carophyllene, humulene, linalool, and methyl nonyl ketone.

In some embodiments, the flavorants employed in the products and methods provided herein are flavorants that are associated with certain foods, such as fruits and nuts that are available locally (referred to as “regionally indigenous”) and/or seasonally and/or which are considered “artisan” foods. Various food items have pleasant and, in some cases, unique, aromatic properties that can advantageously employed within the disclosed products. In particular, in some embodiments, such food items do not have wide distribution (e.g., as they are only produced/grown in a limited area, have very short shelf life, and/or are only produced/grown during a limited season of the year). Although not intending to be limiting, certain examples of such food items are paw paws (tropical fruits native to the eastern United States), persimmons, black walnuts, scuppernongs, muscadines, and elderberries. According to the present disclosure, components of such foods or synthetically produced compounds associated with the one or more such components can be incorporated within the disclosed products to endow them with beneficial sensory characteristics.

In certain embodiments, articles are provided which incorporate one or more flavorants associated with fruits of the asimina triloba tree (also referred to herein as “pawpaws”). The pawpaw is a small deciduous tree native to the eastern United States and Canada. The pawpaw fruit is a large, yellowish-green to brown fruit. Pawpaw fruits generally have a strong aroma, with a sweet, custardish flavor, somewhat similar to banana, mango, pineapple, and/or cantaloupe. There are over 80 varieties of pawpaws, with more than 40 named varieties of pawpaw fruits, each may have different characteristics, e.g., different flavors and aromas. The pawpaw fruit has been found to contain high levels of phenols and flavonoids, with volatile components attributable to the aroma including mainly ethyl and methyl esters of fatty acids (e.g., ethyl hexanoate, ethyl butanoate, methyl hexanoate, methyl octanoate, and/or ethyl octanoate).

One or more compounds (including at least one compound that provides desirable sensory characteristics) extracted from the pawpaw fruit can, in some embodiments, be incorporated within the disclosed product. As described above with respect to hops, an extract of the pawpaw fruit can be provided for inclusion within products according to the present disclosure. Methods for providing pawpaw fruit pulp are widely known and, in some embodiments, such pulp can be pressed and filtered or extracted to obtain a liquid suitable for incorporation within the disclosed products.

In other embodiments, synthetic versions of one or more aroma compounds associated with the pawpaw fruit can be prepared and incorporated within the disclosed products. For example, in some embodiments, one or more of ethyl hexanoate, ethyl butanoate, methyl hexanoate, methyl octanoate and ethyl octanoate are incorporated within the disclosed products, and typically, such products will include ethyl hexanoate (understood to contribute significantly to the aroma of the pawpaw fruit). The use of synthetic compounds in this manner can address issues with respect to limited availability of the pawpaw fruit, providing sensory characteristics reminiscent of the pawpaw fruit year-round and in all geographic locations. Typically, although not limited thereto, to mimic the unique aroma of pawpaw fruit (or other food items), a mixture of two or more synthetic chemical compounds is provided.

Such processes can be similarly applied for other food items, e.g., fruits and nuts, particularly those with limited availability as referenced above. As such, extracts/liquids produced from other food items can be employed in the products disclosed herein or synthetically prepared compounds associated with the taste or aroma of such food items can be used in the disclosed products to mimic the sensory characteristics thereof.

For example, persimmons have been described as having an aroma indicative of fresh peach, orange, sweet, woody, floral, green, and/or potato. Persimmon extracts can be prepared or obtained (as persimmon extracts are commercially available) and can be employed for this purpose. Alternatively, compounds associated with the sensory characteristics of persimmons (e.g., methional, (E)-2-hexenal, phenylacetaldehyde, (E,Z)-2,6-nonadienal, hexanal, furaneol, (E,E,Z)-2,4,6-nonatrienal and (E,Z,Z)-2,4,7-decatrienal) can be synthesized or obtained for inclusion within the disclosed products to mimic the aroma, of persimmons.

Black walnuts have been described as having an aroma and taste that is nutty, bold and smoky. Black walnut extracts can be prepared or obtained (as black walnut extracts are commercially available) and can be employed for this purpose. Alternatively, compounds associated with the sensory characteristics of black walnuts (e.g., carbonyls, alcohols, and terpenes) can be synthesized or obtained for inclusion within the disclosed products to mimic the aroma of black walnuts.

Scuppernongs and muscadines have been described as having intense aromas, e.g., a musky aroma. Scuppernong and muscadine extracts can be prepared or obtained and can be employed for this purpose. Scuppernongs can be treated, e.g., so as to obtain steam volatile oils. See, e.g., J. Food Sci. 49 (1): 64-66, January 1984, which is incorporated herein by reference. Alternatively, compounds associated with the sensory characteristics of scuppernongs (e.g., aldehydes, substituted furans, ketones, alcohols, aromatic and aliphatic hydrocarbons, terpenes, and esters, such as 2-pentanone, hexanal, toluene, furfural, 2-hexenal, xylene, benzaldehyde, geraniol, β-phenylethanol formate, γ-terpene, benzylalcohol, phenylethanol, linalool, nonadienal, and/or β-ionone) can be synthesized or obtained for inclusion within the disclosed products to mimic the aroma of scuppernongs. Muscadines can be treated, e.g., via solid phase extraction to obtain an extract. See, e.g., Food Sci. BioTech 25 (5): 1319-1326, October 2016, which is incorporated herein by reference. Alternatively, one or more of the compounds associated with the sensory characteristics of muscadines (e.g., butyl-2-butenoate, hexyl acetate, propyl acetate, ethyl trans-2-butenoate, hexyl-2-butenoate, ethyl acetate, butyl acetate, 1-octanol, ethyl hexanoate, β-citral, nonanal, decanal, β-citronellol, myrcenol, β-ocimene, and 1-limonene) can be synthesized or obtained for inclusion within the disclosed products to mimic the aroma of muscadines.

Elderberries have been described as exhibiting aromas such as fruity/sweet. Elderberry extracts can be prepared or obtained and can be employed for this purpose. For example, elderberries can be air-dried and crushed and subjected to distillation. See, e.g., Nat. Vol. & Essent. Oils 1 (1): 51-54 (2004), which is incorporated herein by reference. Alternatively, compounds associated with the aroma of elderberries (e.g., (E)-damascenone, dihydroedulan, ethyl-9-decenoate, 2-phenyl ethanol, phenylacetaldehyde, nonanal, and aliphatic esters such as ethyl 2-methylbutanoate, ethyl 3-methylbutanoate, methyl heptanoate, methyl octanoate, and methyl nonanoate).

The plant-derived flavorant material (e.g., solid, extract, or oil) and/or synthetic flavorant compound(s) described herein above can be incorporated within various types of products, as will be detailed herein. The material can be used, for example, as a component of tobacco products, such as a component of tobacco burning products (e.g., cigarettes, cigars, pipe tobaccos, and the like), tobacco heating smoking articles (e.g., cigarettes such as those sold under the brand name Eclipse by R. J. Reynolds Tobacco Company), smokeless tobacco products (e.g., moist snuff, chewing tobacco, snus and so-called dissolvable tobacco products), so-called electronic cigarettes, and the like.

For example, in some embodiments, the disclosed flavorants are incorporated within smoking articles. Typically, the smoking articles are sufficiently compact to be considered “hand-held” devices. As used herein, the term “smoking article” is intended to mean an article that provides many of the sensations (e.g., inhalation and exhalation rituals, types of tastes or flavors, organoleptic effects, physical feel, use rituals, visual cues such as those provided by visible aerosol, and the like) of smoking a cigarette, cigar, or pipe without any substantial degree of combustion of any component of the article. As used herein, the term “smoking article” does not necessarily mean that, in operation, the article produces smoke in the sense of the aerosol resulting from by-product of combustion or pyrolysis of tobacco, but rather, that the article yields vapors (including vapors within aerosols that can be considered to be visible aerosols that might be considered to be described as smoke-like) resulting from volitization or vaporization of certain components of the article or device. In some embodiments, articles characterized as smoking articles incorporate tobacco and/or components derived from tobacco (e.g., including, but not limited to, nicotine).

In some embodiments, the articles provided according to the present disclosure can be characterized as being vapor-producing articles, aerosolization articles, or medicament delivery articles. Thus, the articles can be arranged so as to provide one or more substances (e.g., flavors and/or pharmaceutical active ingredients) in an inhalable form or state. For example, inhalable substances can be substantially in the form of a vapor (i.e., a substance that is in the gas phase at a temperature lower than its critical point). Alternatively, inhalable substances can be in the form of an aerosol (i.e., a suspension of fine solid particles or liquid droplets in a gas). For purposes of simplicity, the term “aerosol” as used herein is meant to include vapors, gases and aerosols of a form or type suitable for human inhalation, whether or not visible, and whether or not of a form that might be considered to be smoke-like.

In use, smoking articles that can be manufactured according to the present disclosure may be subjected to many of the physical actions of an individual in using a traditional type of smoking article (e.g., a cigarette, cigar or pipe that is employed by lighting with a flame and used by inhaling tobacco that is subsequently burned). For example, the user of a smoking article of the present disclosure can hold that article much like a traditional type of smoking article, draw on one end of that article for inhalation of aerosol produced by that article, take puffs at selected intervals of time.

In one embodiment, the plant-derived flavorant material or synthetic compound(s) described herein above are incorporated within a traditional electronic cigarette. Various types and configurations of electronic cigarettes are known and can be adapted so as to incorporate an oil, extract, or synthetic compound(s) as disclosed herein. Some exemplary designs and component arrangements can be appreciated upon consideration of the commercially available electronic smoking articles, such as those representative products listed in the background of the present disclosure.

Typical electronic cigarettes include some combination of power source (i.e., an electrical power source), at least one control component (e.g., means for actuating, controlling, regulating and ceasing power for heat generation, such as by controlling electrical current flow the power source to other components of the article), a heater or heat generation component (e.g., an electrical resistance heating element or component commonly referred to as an “atomizer”), and an aerosol precursor component (e.g., commonly a liquid capable of yielding an aerosol upon application of sufficient heat, such as ingredients commonly referred to as “smoke juice,” “e-liquid” and “e-juice”), and a mouthend region or tip for allowing draw upon the smoking article for aerosol inhalation (e.g., a defined air flow path through the article such that aerosol generated can be withdrawn therefrom upon draw).

Alignment of the components within the article can vary. In specific embodiments, the aerosol precursor component can be located near an end of the article (e.g., with a cartridge, which in certain circumstances can be replaceable and disposable) that is proximal to the mouth of a user so as to maximize aerosol delivery to the user. Other configurations, however, are not excluded. Generally, the heater component can be positioned sufficiently near that aerosol precursor component so that heat from the heater component can volatilize the aerosol precursor (as well as one or more flavorants, medicaments, or the like that may likewise be provided for delivery to a user) and form an aerosol for delivery to the user. When the heating member heats the aerosol precursor component, an aerosol is formed, released, or generated in a physical form suitable for inhalation by a consumer. It should be noted that the foregoing terms are meant to be interchangeable such that reference to release, releasing, releases, or released includes form or generate, forming or generating, forms or generates, and formed or generated. Specifically, an inhalable substance is released in the form of a vapor or aerosol or mixture thereof. Additionally, the selection of various smoking article components can be appreciated upon consideration of the commercially available electronic smoking articles, such as those representative products listed in the background art section of the present disclosure.

The aerosol precursor component, which may also be referred to as a vapor precursor composition, can comprise one or more different components. For example, the aerosol precursor can include a polyhydric alcohol (e.g., glycerin, propylene glycol, or a mixture thereof). Representative types of further aerosol precursor compositions are set forth in U.S. Pat. No. 4,793,365 to Sensabaugh, Jr. et al.; U.S. Pat. No. 5,101,839 to Jakob et al.; PCT WO 98/57556 to Biggs et al.; and Chemical and Biological Studies on New Cigarette Prototypes that Heat Instead of Burn Tobacco, R. J. Reynolds Tobacco Company Monograph (1988); the disclosures of which are incorporated herein by reference. Aerosol precursor compositions can include further liquid materials, such as water. For example, aerosol precursor compositions can incorporate mixtures of glycerin and water, or mixtures of propylene glycol and water, or mixtures of propylene glycol and glycerin, or mixtures of propylene glycol, glycerin, and water. Exemplary aerosol precursor compositions also include those types of materials incorporated within devices available through Atlanta Imports Inc., Acworth, Ga., USA., as an electronic cigar having the brand name E-CIG, which can be employed using associated Smoking Cartridges Type C1la, C2a, C3a, C4a, C1b, C2b, C3b and C4b; and as Ruyan Atomizing Electronic Pipe and Ruyan Atomizing Electronic Cigarette from Ruyan SBT Technology and Development Co., Ltd., Beijing, China.

The aerosol precursor composition used in the disclosed smoking article further can comprise one or more medicaments or other inhalable materials. Particularly, an inhalable substance delivered using a smoking article according to the present invention can comprise a tobacco component or a tobacco-derived material. For aerosol delivery systems that are characterized as electronic cigarettes, the aerosol precursor composition most preferably incorporates tobacco or components derived from tobacco. In one regard, the tobacco may be provided as parts or pieces of tobacco, such as finely ground, milled or powdered tobacco lamina. In another regard, the tobacco may be provided in the form of an extract, such as a spray dried extract that incorporates many of the water soluble components of tobacco. Alternatively, tobacco extracts may have the form of relatively high nicotine content extracts, which extracts also incorporate minor amounts of other extracted components derived from tobacco. In one regard, a component that is derived from tobacco, and that may be employed in a highly purified or essentially pure form, is nicotine (e.g., pharmaceutical grade nicotine). In some embodiments, nicotine is provided as a synthetic compound for use in the disclosed articles. In some embodiments, nicotine is provided in salt form, e.g., as described in U.S. Pat. No. 2,033,909 to Cox et al. and U.S. Pat. No. 9,738,622 to Dull et al. and Perfetti, Beitrage Tabakforschung Int., 12, 43-54 (1983), which are incorporated herein by reference in their entireties.

Advantageously, the disclosed plant-derived flavorant and/or synthetic flavorant compounds described herein above are incorporated within the aerosol precursor to provide unique sensory characteristics (e.g., aroma and/or flavor) to the consumer during use (i.e., when the aerosol precursor is heated, as referenced above). Such flavorants may, in some embodiments, be the sole source of flavorant in the smoking article. For example, a smoking article (or cartridge for use therein) can be specifically designed so as to exhibit hops aroma/flavor, to exhibit pawpaw fruit aroma/flavor, to exhibit persimmon aroma/flavor, to exhibit black walnut aroma/flavor, to exhibit scuppernong aroma/flavor, to exhibit muscadine aroma/flavor, to exhibit elderberry aroma/flavor, or to exhibit some combination of such aromas/flavors. In other embodiments, a plant-derived flavorant or synthetic flavorant compound according to the present disclosure is used in combination with other flavorants, such as those flavorants commonly employed in such articles.

Such additional flavoring agents can be provided from tobacco or from sources other than tobacco, can be natural or artificial in nature, and can be employed as concentrates or flavor packages. Exemplary additional flavoring agents include vanillin, ethyl vanillin, cream, tea, coffee, fruit (e.g., apple, cherry, strawberry, peach and citrus flavors, including lime and lemon), maple, menthol, mint, peppermint, spearmint, wintergreen, nutmeg, clove, lavender, cardamom, ginger, honey, anise, sage, cinnamon, sandalwood, jasmine, cascarilla, cocoa, licorice, and flavorings and flavor packages of the type and character traditionally used for the flavoring of cigarette, cigar, and pipe tobaccos. Syrups, such as high fructose corn syrup, also can be employed. Such additional flavoring agents also can include acidic or basic characteristics (e.g., organic acids, such as levulinic acid, succinic acid, lactic acid, and pyruvic acid). The flavoring agents can be combined with the aerosol-generating material if desired. Exemplary plant-derived compositions that may be used are disclosed in US App. Pub. No. 2012/0152265 to Dube et al. and U.S. Pat. No. 9,107,453 to Dube et al., the disclosures of which are incorporated herein by reference in their entireties.

Aerosol precursor compositions also may include ingredients that exhibit acidic or basic characteristics (e.g., organic acids, ammonium salts or organic amines). For example, certain organic acids (e.g., levulinic acid, succinic acid, lactic acid, and pyruvic acid) may be included in an aerosol precursor formulation incorporating nicotine, preferably in amounts up to being equimolar (based on total organic acid content) with the nicotine. For example, the aerosol precursor may include about 0.1 to about 0.5 moles of levulinic acid per one mole of nicotine, about 0.1 to about 0.5 moles of succinic acid per one mole of nicotine, about 0.1 to about 0.5 moles of lactic acid per one mole of nicotine, about 0.1 to about 0.5 moles of pyruvic acid per one mole of nicotine, or various permutations and combinations thereof, up to a concentration wherein the total amount of organic acid present is equimolar to the total amount of nicotine present in the aerosol precursor composition. However, in some aspects of the present disclosure, the aerosol precursor composition is free of any acidic (or basic) characteristics or additives.

Although, in some embodiments, the disclosed plant-derived or synthetic flavorant(s) disclosed herein are incorporated within the aerosol precursor composition, such flavorants can be incorporated anywhere within the smoking article where aerosol is generated therefrom. In some embodiments, these components (alone, or in combination with one or more of the other inhalable components referenced above) can be provided, e.g., in a reservoir. In some embodiments, such components can be supplied directly to the resistive heating element or may be provided on a substrate. As such, defined aliquots of the disclosed flavorants (and/or other inhalable material) may, in some embodiments, be separately or simultaneously delivered to the resistive heating element to release the flavor, medicament, or other inhalable material into an air stream to be inhaled by a user along with the further components of the aerosol precursor or vapor precursor composition.

As one non-limiting example, a representative aerosol precursor composition or substance can include one or more of the plant-derived flavorants or synthetic flavorant compounds disclosed herein, glycerin, propylene glycol, water, saline, and nicotine, and combinations or mixtures of any or all of those components. For example, in one instance, a representative aerosol precursor composition may include (on a weight basis), in addition to the one or more of the plant-derived flavorants or synthetic flavorant compounds disclosed herein, about 70% to about 100% glycerin, and often about 80% to about 90% glycerin; about 5% to about 25% water, often about 10% to about 20% water; and about 0.1% to about 5% nicotine, often about 2% to about 3% nicotine. In one particular non-limiting example, a representative aerosol precursor composition may include, in addition to one or more of the plant-derived flavorants or synthetic flavorant compounds disclosed herein, about 84% glycerin, about 14% water, and about 2% nicotine. The representative aerosol precursor composition may also include propylene glycol, optional flavoring agents or other additives in varying amounts on a weight basis. In some instances, the aerosol precursor composition may comprise, in addition to the one or more of the plant-derived flavorants or synthetic flavorant compounds disclosed herein, up to about 100% by weight of any of glycerin, water, and saline, as necessary or desired.

The amount of one or more of the plant-derived flavorants or synthetic flavorant compounds disclosed herein incorporated within the aerosol precursor composition can vary and is dependent upon, e.g., the strength of the flavor/aroma desired, the form (i.e., solid, oil, extract, or compound(s)) included, and the specific properties exhibited by the plant-derived flavorants or synthetic flavorant compounds. Such amounts could be readily adjusted to achieve the desired level of organoleptic enhancement desired with respect to use of the smoking article.

Representative types of aerosol precursor components and formulations (into which the disclosed plant-derived flavorants or synthetic flavorant compounds can be incorporated) also are set forth and characterized in U.S. Pat. No. 7,217,320 to Robinson et al. and U.S. Pat. Pub. Nos. 2013/0008457 to Zheng et al.; 2013/0213417 to Chong et al. and 2014/0060554 to Collett et al., the disclosures of which are incorporated herein by reference. Other aerosol precursors that may be employed include the aerosol precursors that have been incorporated in the VUSE® product by R. J. Reynolds Vapor Company, the BLU™ product by Lorillard Technologies, the MISTIC MENTHOL product by Mistic Ecigs, and the VYPE product by CN Creative Ltd. Also desirable are the so-called “smoke juices” for electronic cigarettes that have been available from Johnson Creek Enterprises LLC.

The amount of aerosol precursor that is incorporated within the aerosol delivery system is such that the aerosol generation arrangement(s) provide acceptable sensory and desirable performance characteristics. For example, it is highly preferred that sufficient amounts of aerosol forming material (e.g., glycerin and/or propylene glycol), be employed in order to provide for the generation of a mainstream aerosol (visible or not visible) that in many regards resembles the appearance of tobacco smoke. The amount of the aerosol precursor composition within the aerosol generation arrangement(s) may be dependent upon factors such as the number of puffs desired per aerosol generation arrangement. Typically, the amount of the aerosol precursor composition incorporated within the aerosol delivery system, and particularly within the aerosol generation arrangement(s), is less than about 2 g, generally less than about 1.5 g, often less than about 1 g and frequently less than about 0.5 g.

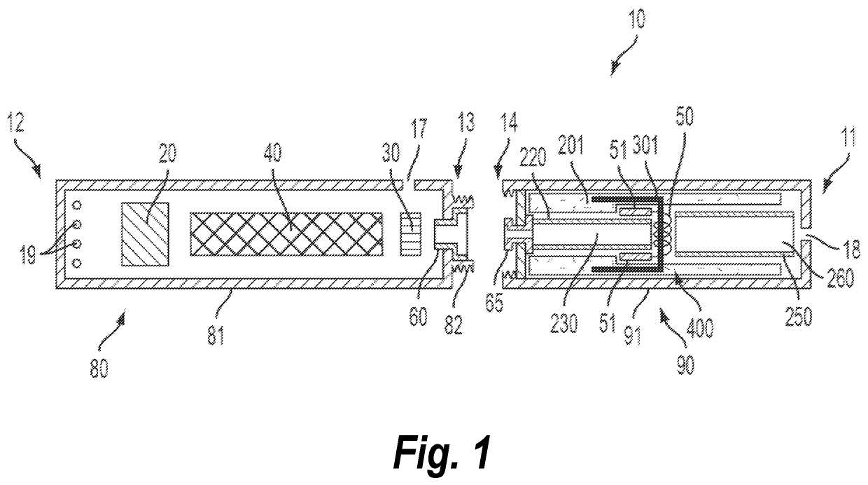

An exemplary smoking article 10 according to the disclosure is shown in FIG. 1. As seen in the surface illustrated therein, the smoking article 10 can comprise a control body 80 and a cartridge 90 that can be aligned in a functioning relationship. In this regard, the control body 80 and the cartridge 90 may be attachable and detachable from each other. Although a threaded engagement is illustrated in FIG. 1, it is understood that further means of engagement are encompassed, such as a press-fit engagement, a magnetic engagement, or the like. The cartridge can particularly include a single use connector as otherwise described herein.

In specific embodiments, the control body 80 may be referred to as being reusable and the cartridge 90 may be referred to as being disposable. In some embodiments, the entire smoking article may be characterized as being disposable in that the control body may be configured for only a limited number of uses (e.g., until a battery power component no longer provides sufficient power to the smoking article) with a limited number of cartridges and, thereafter, the entire smoking article 10, including the control body, may be discarded. In other embodiments, the control body may have a replaceable battery such that the control body can be reused through a number of battery exchanges and with many cartridges. Similarly, the smoking article 10 may be rechargeable and thus may be combined with any type of recharging technology, including connection to a typical electrical outlet, connection to a car charger (i.e., cigarette lighter receptacle), and connection to a computer, such as through a USB cable.

The control body 80 includes a control component 20, a flow sensor 30, and a battery 40. Although these components are illustrated in a specific alignment, it is understood that various alignments of the components are encompassed by the present disclosure. The control body 80 further includes a plurality of indicators 19 at a distal end 12 of the control body shell 81. Such indicators 19 can show the number of puffs taken or remaining from the smoking article can be indicative of an active or inactive status, can light up in response to a puff, or the like. The indicators can be provided in varying numbers and can take on different shapes and can even be simply an opening in the body (such as for release of sound when such indicators are present).

Various positions for one or more air intakes 17 are encompassed by the present disclosure. As shown, the air intake 17 may be positioned in the control body shell 81 such that air drawn through the intake sufficiently contacts the flow sensor 30 to activate the sensor (although other positions are encompassed, particularly if different sensing means are provided or if manual actuation, such as with a push button, is provided). A receptacle 60 also is included at the proximal attachment end 13 of the control body 80 and extends into the control body projection 82 to allow for ease of electrical connection with the resistive heating element 50 when the cartridge 90 is attached to the control body. In the illustrated embodiment, the receptacle 60 includes a central open passage to facilitate air flow from the air intake in the control body into the cartridge during use of the article 10.

The cartridge 90 includes a cartridge shell 91 with a mouth opening 18 at the mouthend 11 thereof to allow passage of air and entrained vapor (i.e., the components of the aerosol precursor composition in an inhalable form) from the cartridge to a consumer during draw on the smoking article 10. The smoking article 10 according to the present disclosure may have an overall shape that may be defined as being substantially rod-like or substantially tubular shaped or substantially cylindrically shaped. As illustrated in FIG. 1, the smoking article 10 has a substantially round cross-section; however, other cross-sectional shapes (e.g., oval, square, triangle, etc.) also are encompassed by the present disclosure. Such language that is descriptive of the physical shape of the smoking article may also be applied to the individual units of the smoking article in embodiments comprising multiple units, such as a control body and a cartridge.

In preferred embodiments, the smoking article 10 may take on a size that is comparative to a cigarette or cigar shape. Thus, the smoking article may have a diameter of about 5 mm to about 25 mm, about 5 mm to about 20 mm, about 6 mm to about 15 mm, or about 6 mm to about 10 mm. Such dimension may particularly correspond to the outer diameter of the control body shell 81 and/or the cartridge shell 91. The control body can have a length of about 50 mm to about 110 mm, about 60 mm to about 100 mm, or about 65 mm to about 95 mm. The cartridge can have a length of about 20 mm to about 60 mm, about 25 mm to about 55 mm, or about 30 mm to about 50 mm. The overall length of the combined cartridge and control body (or the overall length of a smoking article according to the disclosure formed of a single, unitary shell) can be approximately equal to or less than the length of a typical cigarette—e.g., about 70 mm to about 130 mm, about 80 mm to about 125 mm, or about 90 mm to about 120 mm.

The cartridge shell 91 of the smoking article 10 can be formed of any material suitable for forming and maintaining an appropriate conformation, such as a tubular shape, and for retaining therein the suitable components of the smoking article. The body can be formed of a single wall, as shown in FIG. 1. The cartridge shell 91 can be formed of a material (natural or synthetic) that is heat resistant so as to retain its structural integrity—e.g., does not degrade—at least at a temperature that is the heating temperature provided by the resistive heating element. In some embodiments, a heat resistant polymer may be used. In other embodiments, the body can be formed from paper, such as a paper that is substantially straw-shaped, or from metal, such as stainless steel. As further discussed herein, the body, such as a paper tube, may have one or more layers associated therewith that function to substantially prevent movement of vapor therethrough. In one example, an aluminum foil layer may be laminated to one surface of the body. Ceramic materials also may be used.

The cartridge 90 further includes a resistive heating element 50 in the form of a metal wire coil. The resistive heating element includes terminals 51 (e.g., positive and negative terminals) at the opposing ends thereof for facilitating current flow through the resistive heating element and for attachment of the appropriate wiring (not illustrated) to form an electrical connection of the resistive heating element with the battery 40 when the cartridge 90 is connected to the control body 80. Specifically, a plug 65 is positioned at the distal attachment end 14 of the cartridge. When the cartridge 90 is connected to the control body 80, the plug 65 engages the receptacle 60 to form an electrical connection such that current controllably flows from the battery 40, through the receptacle and plug, and to the resistive heating element 50. The cartridge shell 91 can continue across the distal attachment end such that this end of the cartridge is substantially closed with the plug protruding therefrom. As illustrated in FIG. 1, the plug 65 includes an open central passage that aligns with the open central passage in the receptacle 60 to allow air to flow from the control body 80 and into the cartridge 90.

Generally, in use, when a consumer draws on the mouthend 11 of the cartridge, the flow sensor 30 detects the change in flow and activates the control component 20 to facilitate current flow through the resistive heating element 50. Thus, it is useful for air flow to travel through the control body 80 in a manner that flow sensor 30 detects air flow almost instantaneously.

The control algorithm may call for power to the resistive heating element 50 to cycle and thus maintain a defined temperature. The control algorithm therefore can be programmed to automatically deactivate the smoking article 10 and discontinue power flow through the smoking article after a defined time lapse without a puff by a consumer. Moreover, the smoking article can include a temperature sensor to provide feedback to the control component. Such sensor can be, for example, in direct contact with the resistive heating element 50. Alternative temperature sensing means likewise may be used, such as relying upon logic control components to evaluate resistance through the resistive heating element and correlate such resistance to the temperature of the element. In other embodiments, the flow sensor 30 may be replaced by appropriate components to provide alternative sensing means, such as capacitive sensing. Still further, one or more control buttons can be included to allow for manual actuation by a consumer to elicit a variety of functions, such as powering the article 10 on and off, turning on the heating element 50 to generate a vapor or aerosol for inhalation, or the like.

When the flow sensor 30 is positioned within the control body 80, it can be useful to have an air intake 17 on the control body. If desired, a sealed flow path can be provided such that the flow sensor 30 within the control body 80 is in fluid connection with the cartridge interior after the cartridge and the control body are engaged, such fluid connection being sealed with respect to the remainder of the components within the control body but opening into the cartridge 90 when attached to the control body. Further, in other embodiments, the flow sensor 30 can be located within the cartridge 90 instead of the control body 80.

A reservoir may utilize a transport element to transport an aerosol precursor composition to an aerosolization zone. As used herein, the term “reservoir” refers to a receptacle or chamber for holding, storing, or retaining a product such as a liquid, fluid, or aerosol. One such example is shown in FIG. 1. As seen therein, the cartridge 90 includes a reservoir layer 201 comprising layers of nonwoven fibers formed into the shape of a tube encircling the interior of the cartridge shell 91, in this embodiment. An aerosol precursor composition is retained in the reservoir layer 201. Liquid components, for example, can be sorptively retained by the reservoir layer 201. The reservoir layer 201 is in fluid connection with a transport element 301 (a wick in this embodiment). The transport element 301 transports the aerosol precursor composition stored in the reservoir layer 201 via capillary action to an aerosolization zone 400 of the cartridge 90. As illustrated, the transport element 301 is in direct contact with the resistive heating element 50 that is in the form of a metal wire coil in this embodiment.

In use, when a user draws on the article 10, the resistive heating element 50 is activated (e.g., such as via a puff sensor), and the components for the aerosol precursor composition are vaporized in the aerosolization zone 400. Drawing upon the mouthend 11 of the article 10 causes ambient air to enter the air intake 17 and pass through the central opening in the receptacle 60 and the central opening in the plug 65. In the cartridge 90, the drawn air passes through an air passage 230 in an air passage tube 220 and combines with the formed vapor in the aerosolization zone 400 to form an aerosol. The aerosol is whisked away from the aerosolization zone, passes through an air passage 260 in an air passage tube 250, and out the mouth opening 18 in the mouthend 11 of the article 10. If desired, the air passage tube 250 can be absent, and an open cavity may reside in the location for formation of aerosol as the aerosol precursor composition is vaporized by the resistive heating element 50.

The smoking article 10 in the embodiment illustrated in FIG. 1 can be characterized as a disposable article. Accordingly, it can be desirable for the reservoir 201 in such embodiments to include a sufficient amount of aerosol precursor composition and any further inhalable materials so that a consumer can obtain more than a single use of the smoking article. For example, the smoking article can include sufficient aerosolizable and/or inhalable materials such that the smoking article can provide a number of puffs substantially equivalent to the number of puffs (of about two seconds duration) available from a plurality of conventional cigarettes—e.g., 2 or more, 5 or more, 10 or more, or 20 or more conventional cigarettes. More particularly, a disposable, single unit article according to the embodiment of FIG. 1 can provide about 20 or more, about 50 or more, or about 100 or more puffs.

Although FIG. 1 is illustrative of a smoking article according to the present disclosure, the scope of the disclosure should not be viewed as being limited to the specific combination and/or arrangement of components illustrated therein. Rather, the present disclosure can encompass a variety of combinations of components useful in forming an electronic smoking article. Reference is made for example to the smoking articles disclosed in U.S. patent application Ser. No. 13/536,438, filed Jun. 28, 2012, and U.S. patent application Ser. No. 13/432,406, filed Mar. 28, 2012, the disclosures of which are incorporated herein by reference in their entirety. Further to the above, representative heating element and materials for use therein are described in U.S. Pat. No. 5,060,671 to Counts et al.; U.S. Pat. No. 5,093,894 to Deevi et al.; U.S. Pat. No. 5,224,498 to Deevi et al.; U.S. Pat. No. 5,228,460 to Sprinkel Jr., et al.; U.S. Pat. No. 5,322,075 to Deevi et al.; U.S. Pat. No. 5,353,813 to Deevi et al.; U.S. Pat. No. 5,468,936 to Deevi et al.; U.S. Pat. No. 5,498,850 to Das; U.S. Pat. No. 5,659,656 to Das; U.S. Pat. No. 5,498,855 to Deevi et al.; U.S. Pat. No. 5,530,225 to Hajaligol; U.S. Pat. No. 5,665,262 to Hajaligol; U.S. Pat. No. 5,573,692 to Das et al.; and U.S. Pat. No. 5,591,368 to Fleischhauer et al., the disclosures of which are incorporated herein by reference in their entireties.

The various components of a smoking article according to the present invention can be chosen from components described in the art and commercially available. Examples of batteries that can be used according to the disclosure are described in US Pub. App. No. 2010/0028766, the disclosure of which is incorporated herein by reference in its entirety.

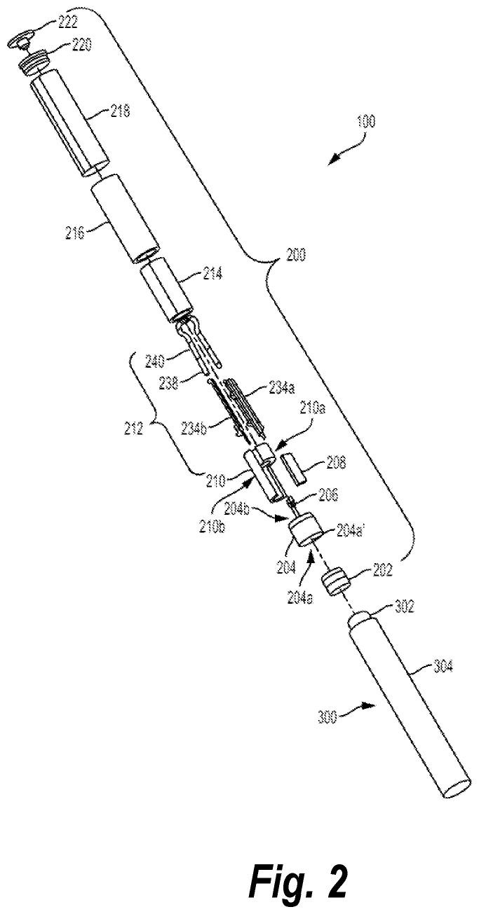

In another embodiment, the disclosed plant-derived flavorants or synthetic flavorant compounds can be employed in a device as disclosed generally in US Pat. Appl. Pub. No. 2015/0335070 to Sears et al., which is incorporated herein by reference. One exemplary such aerosol-delivery device is depicted, for example, in FIG. 2, and is described generally herein below. In particular, FIG. 2 illustrates a partially exploded view of an aerosol delivery system 100 including a cartridge body 200 and a control body 300 (otherwise referred to herein as “cartridge body portion” and “control body portion,” respectively). The cartridge body 200 and the control body 300 can be permanently or detachably aligned, or removably engaged, in a functioning relationship. Various mechanisms may be used to connect the cartridge body 200 to the control body 300 to result in a threaded engagement, a press-fit engagement, an interference fit, a magnetic engagement, or the like. The aerosol delivery system 100 may be substantially rod-like, substantially tubular shaped, or substantially cylindrically shaped in some embodiments, when the cartridge body 200 and the control body 300 are in an assembled configuration. As used herein, “tubular” is intended to refer to a hollow, elongated body, but is not limited to a specific cross-sectional shape or to a specific outer contour of the body. One skilled in the art will also appreciate that, in some instances and though not described in detail herein, the cartridge body 200 and the control body 300 forming the aerosol delivery system 100 may be configured in a single-piece, non-detachable form and may incorporate the components, aspects, and features associated with and disclosed in the present disclosure.

In some instances, one or both of the cartridge body 200 and the control body 300 may be referred to as being disposable (i.e., the single piece, non-detachable form previously disclosed) or as being reusable. For example, a reusable control body 300 may have a replaceable battery or a rechargeable battery and thus may be combined with any type of recharging technology, including connection to a typical alternating current electrical outlet, connection to a car charger (i.e., cigarette lighter receptacle), and connection to a computer, such as through a universal serial bus (USB) cable. In general, an aerosol delivery system of the type disclosed herein incorporates a battery or other electrical power source to provide current flow sufficient to provide various functionalities to the article, such as powering of a heater or heating element, powering of control systems, powering of indicators, and the like. The power source can take on various embodiments. Preferably, the power source is able to deliver sufficient power to rapidly heat the heating element to provide for aerosol formation and power the article through use for the desired duration of time. The power source preferably is sized to fit conveniently within the aerosol delivery device/system so that the aerosol delivery device/system can be easily handled; and additionally, a preferred power source is of a sufficiently light weight to not detract from a desirable smoking experience. Further, in some instances, the cartridge body 200 may comprise a single-use cartridge (i.e., disposable), as disclosed, for example, in U.S. Pat. App. Pub. No. 2014/0060555 to Chang et al., which is incorporated herein by reference in its entirety. Various modifications to the structure of the smoking article can be envisioned and are encompassed herein, e.g., as disclosed in US Pat. Appl. Pub. No. 2015/0335070 to Sears et al., which is incorporated herein by reference. Still further features, controls or components that can be incorporated into aerosol delivery devices and systems of the present disclosure are described in U.S. Pat. No. 5,967,148 to Harris et al.; 5,934,289 to Watkins et al.; U.S. Pat. No. 5,954,979 to Counts et al.; U.S. Pat. No. 6,040,560 to Fleischhauer et al.; U.S. Pat. No. 7,726,320 to Robinson et al.; U.S. Pat. No. 8,365,742 to Hon; U.S. Pat. Nos. 8,402,976 and 8,689,804 to Fernando et al.; U.S. Pat. App. Pub. Nos. 2013/0192623 to Tucker et al.; 2013/0298905 to Leven et al.; 2013/0180553 to Kim et al. and 2014/0000638 to Sebastian et al.; and U.S. Pat. App. Pub. Nos. 2014/0261495 to Novak, III et al. and 2014/0261408 to DePiano et al.; which are incorporated herein by reference in their entireties.

The cartridge body 200 of FIG. 2 is illustrated in an exploded configuration. As illustrated, the cartridge body 200 may comprise a base shipping plug 202, a base 204, a control component terminal 206, an electronic control component 208, a flow tube 210, an atomizer 212, a reservoir substrate 214, an outer body 216, a label 218, a mouthpiece 220, and a mouthpiece shipping plug 222 according to an example embodiment of the present disclosure. The base 204 may be coupled to a first end of the outer body 216 and the mouthpiece 220 may be coupled to an opposing second end of the outer body 216 to enclose the remaining components of the cartridge body 200 therein. The base 204 may be configured to removably engage the coupler 302 of the control body 300. In some instances, the base 204 may comprise anti-rotation features that substantially prevent relative rotation between the cartridge body and the control body as disclosed in U.S. Pat. App. Pub. No. 2014/0261495 to Novak, III et al., which is incorporated herein by reference in its entirety. Various representative coupling mechanisms for upstream and downstream components of electronic cigarettes have been set forth in the patent literature and have been employed for the production of commercially available electronic cigarettes. For example, representative types of coupling mechanisms and components for electronic cigarettes are described in U.S. Pat. App. Pub. No. 2014/0261495 to Novak, III et al., and 2015/0216232, to Bless et al., which are incorporated herein by reference.

The base shipping plug 202 may be configured to engage and protect the base 204 prior to use of the cartridge body 200. Similarly, the mouthpiece shipping plug 222 may be configured to engage and protect the mouthpiece 220 prior to use of the cartridge body 200. The control component terminal 206, the electronic control component 208, the flow tube 210, the atomizer 212, and the reservoir substrate 214 (engaging the aerosol precursor composition or substance) may be retained within the outer body 216. The label 218 may at least partially surround the outer body 216 and include information such as a product identifier thereon.

Alignment of the components within either or both of the control body and the cartridge body of the aerosol delivery device/system can vary. In particular aspects, the aerosol precursor composition can be located near one end of the overall article (e.g., within a cartridge body, which in certain circumstances can be replaceable and disposable), which may be configured to be positioned in relatively closer proximity to the mouth of a user so as to maximize aerosol delivery to the user. Other configurations, however, are not excluded. Generally, the heating element can be positioned sufficiently near the aerosol precursor composition so that heat from the heating element can volatilize the aerosol precursor (and/or one or more flavorants, medicaments, or the like that may likewise be provided for delivery to a user) and form an aerosol for delivery to the user. When the heating element heats the aerosol precursor composition, an aerosol is formed, released, or generated in a physical form suitable for inhalation by a consumer. It should be noted that the foregoing terms are meant to be interchangeable such that reference to release, releasing, releases, or released includes form or generate, forming or generating, forms or generates, and formed or generated. Specifically, an inhalable substance is released in the form of a vapor or aerosol or mixture thereof. Additionally, the selection of various aerosol delivery device components can be appreciated upon consideration of the commercially available electronic aerosol delivery devices, such as those representative products listed above in the present disclosure.

The atomizer (i.e., an aerosol generation arrangement) 212 may comprise a first heating terminal 234a and a second heating terminal 234b, a liquid transport element 238 and a heating element 240. In this regard, the reservoir and/or reservoir substrate 214 may be configured to hold an aerosol precursor composition. The aerosol precursor composition, also referred to as a vapor precursor composition, may comprise a variety of components, as referenced above. Such components may include, by way of example, any of a polyhydric alcohol (e.g., glycerin, propylene glycol, or a mixture thereof), nicotine, tobacco, tobacco extract, water, flavorants, and combinations thereof. As described above, in some embodiments, the aerosol precursor composition comprises one or more plant-derived flavorants or synthetic flavorant compounds disclosed herein.

The reservoir substrate 214 may comprise a plurality of layers of nonwoven fibers formed into the shape of a tube encircling the interior of the outer body 216 of the cartridge body 200. Thus, liquid components, for example, can be sorptively retained by the reservoir substrate 214. The reservoir substrate 214 is in fluid connection with the liquid transport element 238. The liquid transport element 238 may be configured to transport liquid (i.e., the aerosol precursor composition) from the reservoir substrate 214 to the heating element 240 via capillary action. Representative types of substrates, reservoirs or other components for supporting the aerosol precursor composition are described in U.S. Pat. No. 8,528,569 to Newton; and U.S. Pat. App. Pub. Nos. 2014/0261487 to Chapman et al and 2015/0059780 to Davis et al.; and U.S. application Ser. No. 14/170,838; filed Feb. 3, 2014, to Bless et al.; which are incorporated herein by reference. Additionally, various wicking materials, and the configuration and operation of those wicking materials within certain types of electronic cigarettes, are set forth in U.S. Pat. App. Pub. No. 2014/0209105 to Sears et al.; which is incorporated herein by reference.

As illustrated, the liquid transport element 238 may be in direct contact with the heating element 240. As further illustrated in FIG. 2, the heating element 240 may comprise a wire defining a plurality of coils wound about the liquid transport element 238. In some instances, the heating element 240 may be formed by winding the wire about the liquid transport element 238 as described in U.S. Pat. App. Pub. No. 2014/0157583 to Ward et al., which is incorporated herein by reference in its entirety. Further, in some instances, the wire may define variable coil spacing, as described in U.S. Pat. App. Pub. No. 2014/0270730 to DePiano et al., which is incorporated herein by reference in its entirety. Various materials configured to produce heat when an electrical current is applied thereto may be employed to form the heating element 240. Example materials from which the wire coil may be formed include Kanthal (FeCrAl), Nichrome, molybdenum disilicide (MoSi₂), molybdenum silicide (MoSi), molybdenum disilicide doped with aluminum (Mo(Si,Al)₂), graphite and graphite-based materials; and ceramic (e.g., a positive or negative temperature coefficient ceramic).

However, various other methods may be employed to form the heating element 240, and various other aspects of heating elements may be employed in the atomizer 212. For example, a stamped heating element may be employed in the atomizer, as described in U.S. Pat. App. Pub. No. 2014/0270729 to DePiano et al., which is incorporated herein by reference in its entirety. Further to the above, additional representative heating elements and materials for use therein are described in U.S. Pat. No. 5,060,671 to Counts et al.; U.S. Pat. No. 5,093,894 to Deevi et al.; U.S. Pat. No. 5,224,498 to Deevi et al.; U.S. Pat. No. 5,228,460 to Sprinkel Jr., et al.; U.S. Pat. No. 5,322,075 to Deevi et al.; U.S. Pat. No. 5,353,813 to Deevi et al.; U.S. Pat. No. 5,468,936 to Deevi et al.; U.S. Pat. No. 5,498,850 to Das; U.S. Pat. No. 5,659,656 to Das; U.S. Pat. No. 5,498,855 to Deevi et al.; U.S. Pat. No. 5,530,225 to Hajaligol; U.S. Pat. No. 5,665,262 to Hajaligol; U.S. Pat. No. 5,573,692 to Das et al.; and U.S. Pat. No. 5,591,368 to Fleischhauer et al., the disclosures of which are incorporated herein by reference in their entireties. Further, chemical heating may be employed in other aspects. A variety of heater components may also be used in particular aspects of the present aerosol delivery device/system. In various instances, one or more microheaters or similar solid state heating elements may be used. Exemplary microheaters that may be utilized are further described herein. Further microheaters and atomizers incorporating microheaters suitable for use in the presently disclosed devices/systems are described in U.S. Pat. App. Pub. No. 2014/0060554 to Collett et al., which is incorporated herein by reference in its entirety.

The first heating terminal 234a and the second heating terminal 234b (e.g., positive and negative terminals) at the opposing ends of the heating element 240 are configured to form an electrical connection (which may be a removable or detachable connection) with the control body 300 when the cartridge body 200 is connected thereto. Further, when the control body 300 is coupled to the cartridge body 200, the electronic control component 208 may form an electrical connection with the control body 300 through the control component terminal 206. The control body 300 may thus employ the electronic control component 208 to determine whether the cartridge 200 is genuine and/or perform other functions. Further, various examples of electronic control components and functions performed thereby are described in U.S. Pat. App. Pub. No. 2014/0096781 to Sears et al., which is incorporated herein by reference in its entirety.

During use, a user may draw on the mouthpiece or mouth-engaging end 220 of the cartridge body 200 of the aerosol delivery system 100. This may pull air through an opening in the control body 300 and/or in the cartridge body 200. For example, in one instance, an opening may be defined between the coupler 302 and the outer body 304 of the control body 300, as described in U.S. Pat. App. Pub. No. 2014/0261408 to DePiano et al., which is incorporated herein by reference in its entirety. However, the flow of air may be received through other parts of the aerosol delivery device/system 100 in other aspects. As noted above, in some aspects the cartridge body 200 may include the flow tube 210. The flow tube 210 may be configured to direct the flow of air received from the control body 300 to the heating element 240 of the atomizer 212.

A sensor in the aerosol delivery device/system 100 (e.g., a puff or flow sensor in the control body 300) may sense the puff. More generally, a sensor or detector may be implemented to control of supply of electric power to the heating element 240 when aerosol generation is desired (e.g., upon draw during use). As such, for example, there is provided a manner or method for turning off the power supply to the heating element 240 when the aerosol generation is not desired during use, and for turning on the power supply to actuate or trigger the generation of heat by the heating element 240 during draw. Additional representative types of sensing or detection mechanisms, structure and configuration thereof, components thereof, and general methods of operation thereof, are described in U.S. Pat. No. 5,261,424 to Sprinkel, Jr.; U.S. Pat. No. 5,372,148 to McCafferty et al.; and PCT WO 2010/003480 by Flick; which are incorporated herein by reference. When the puff is sensed, the control body 300 may direct current to the heating element 240 through a circuit including the first heating terminal 234a and the second heating terminal 234b. Accordingly, the heating element 240 may vaporize the aerosol precursor composition directed to an aerosolization zone from the reservoir substrate 214 by the liquid transport element 238. Thus, the mouthpiece 220 may allow passage of air and entrained vapor (i.e., the components of the aerosol precursor composition in an inhalable form, for example, as an aerosol) from the cartridge body 200 to a consumer drawing thereon. Various other details with respect to the components that may be included in the cartridge body 200, are provided, for example, in U.S. Pat. App. Pub. No. 2014/0261495 to Novak, III et al., which is incorporated herein by reference in its entirety.

Various components of an aerosol delivery device/system can be chosen from components described in the art and commercially available. Reference is made for example to the reservoir and heater system for controllable delivery of multiple aerosolizable materials in an electronic smoking article disclosed in U.S. Pat. App. Pub. No. 2014/0000638 to Sebastian et al., which is incorporated herein by reference in its entirety. Note further that portions of the cartridge body 200 illustrated in FIG. 1 are optional. In this regard, by way of example, the cartridge body 200 may not necessarily include the flow tube 210, the control component terminal 206, and/or the electronic control component 208, in some instances.

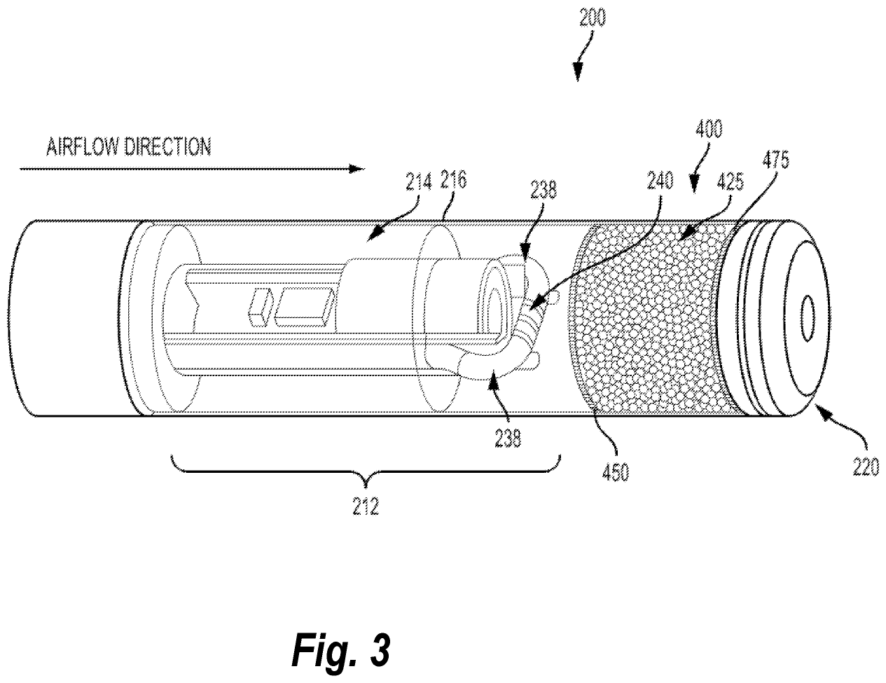

One particular aspect of the present disclosure is illustrated, for example, in FIG. 3. In such instances, the cartridge body 200 may further incorporate a second aerosol generation arrangement 400 (the atomizer 212 being considered “a first aerosol generation arrangement”) disposed in the outer body 216, longitudinally between the atomizer 212 and the mouthpiece or mouth-engaging end 220 of the cartridge body 200. In some aspects, the second aerosol generation arrangement 400 is generally porous or otherwise configured to allow the passage of air therethrough. In some particular instances, the second aerosol generation arrangement 400 may include one or more aerosol-generating elements 425 that may be comprised of at least one or a plurality of pellets or beads or other appropriate elements or combinations thereof. In some instances, the at least one or a plurality of pellets or beads or other appropriate elements or combinations thereof forming the aerosol-generating element(s) 425 may be coaxially circumscribed by a generally tubular-shaped heat conductive member (not shown), if necessary, and/or circumscribed or otherwise jacketed by insulation (e.g., a non-woven mat or layer of glass filaments or fibers), or other suitable material (not shown).

The overall configuration of the second aerosol generation arrangement 400 within the cartridge body 200 of the aerosol delivery device/system 100 can be considered to be generally cylindrical in nature. Representative preferred beads or other objects may be produced from a formulation that incorporates one or more of the flavorant materials disclosed herein (e.g., solid hops, hop-derived oils, hop-derived extracts, fruit/nut-derived extracts, and/or synthetic compounds disclosed herein. The beads most preferably incorporate such components and a visible or non-visible aerosol forming material (e.g., glycerin or other material that generates a visible vapor that resembles smoke). That is, components of the beads are preferably configured to act as substrate components for volatile flavors, vapor forming materials, moisture or other liquid(s), and/or aerosol forming materials that are carried thereby.

The beads can, in some embodiments, further comprise tobacco (e.g., particulate tobacco), components of tobacco and/or materials that are otherwise derived from tobacco (e.g., tobacco extracts such as aqueous tobacco extracts or nicotine derived from tobacco including pharmaceutical grade nicotine). In some aspects, the aerosol-generating element(s) 425 may include or otherwise comprise or be configured as, for example, marumarized tobacco beads of varying shapes and sizes, a monolith of bonded (e.g., sintered) beads; a porous monolith; a single porous structure; a honeycomb monolith; a single piece of a porous material; beads of extruded tobacco; beads of porous material containing tobacco extract (e.g., calcium carbonate, ceramic, or the like); reconstituted tobacco shreds; expanded tobacco shreds; extruded rods of various materials (including hollow cylinders and slotted rods) containing tobacco flavors; shavings, granules, capsules, and/or microcapsules of various materials containing tobacco flavors or other substances, whether in a liquid or other form; and treatments or combinations thereof.

In general, as used herein, the terms “pellets” and “beads” are meant to include beads, pellets, or other discrete small units or pieces of that may include (in addition to those otherwise disclosed herein), for example, carbon pieces, extruded carbon pieces cut into pellets, ceramic beads, marumarized tobacco pieces, and the like, or combinations thereof. For example, granules, pellets or beads can be generally cylindrical or spherical extruded or compressed granules, pellets or beads comprised of a moistened mixture or slurry of milled tobacco lamina, fillers (e.g., granular calcium carbonate), flavors, visible aerosol forming materials and binders (e.g., carboxy methylcellulose) that are formed, cut or spun to the desired size and shape, and then dried to retain the desired configuration. However, such “pellets” or “beads” may comprise any suitable elements, or combination of elements, meeting the preferred aspects as disclosed herein. For example, some or all of the beads or pellets can comprise spherical capsules that are heat sensitive, so that when included in the aerosol-generating element and exposed to heat, the rupture or decomposition thereof causes the release of glycerin, propylene glycol, water, saline, tobacco flavor and/or nicotine or other substances or additives. Also, the beads can comprise ceramic or absorbent clay or silica or absorbent carbon to hold and release an aerosol former. Further, in some aspects, the beads/pellets may comprise a heat conductive material such as, for example, heat conductive graphite, heat conductive ceramic, a metal, tobacco cast on foil, a metal or other suitable material impregnated with appropriate aerosol-generating substances such as glycerin and flavor(s), or a suitable cast sheet material appropriately formed into the desired beads/pellets.

In one particular example, the beads/pellets (particles) may be comprised, by weight, of about 20 to about 70% water, about 10 to about 50% glycerin, and 0 to about 5% of a binder (preferably carboxymethylcellulose, guar gum, potassium, or ammonium alginate), further comprising an additional amount of the flavorant material (i.e., plant-derived flavorant and/or synthetic flavorant compound(s)) disclosed herein. The amount of flavorant material depends, e.g., on the particular type and form of the flavorant material incorporated. For example, a greater amount of solid hops material is typically required to provide a comparable effect as a hop oil or concentrated hop extract. In one particular embodiment, hops are provided within a bead in solid (milled form), at a concentration of from about 20% to 70% by weight based on the entirety of the bead).

The particles may, in some embodiments, be compressed to hold the glycerol and, upon compression, may form a porous matrix that facilitates migration of the aerosol generating components to promote efficient aerosol formation. The manner by which the aerosol forming material is contacted with the substrate material can vary. The aerosol forming material can be applied to a formed material, can be incorporated into processed materials during manufacture of those materials, or can be endogenous to that material. Aerosol-forming material, such as glycerin, can be dissolved or dispersed in an aqueous liquid, or other suitable solvent or liquid carrier, and sprayed onto that substrate material. See, for example, U.S. Patent Appl. Pub. No. 2005/0066986 to Nestor et al. and 2012/0067360 to Conner et al.; which are incorporated herein by reference. The calcium carbonate or other inorganic filler assists in creating porosity within the particles, and may also function to absorb heat which may, in some instances limit or otherwise prevent scorching of the aerosol generating components, as well as assisting in and promoting aerosol formation. See also, for example, those types of materials set forth in U.S. Pat. No. 5,105,831 to Banerjee, et al., and U.S. Pat. App. Pub. Nos. 2004/0173229 to Crooks et al.; 2011/0271971 to Conner et al.; and 2012/0042885 to Stone et al.; which are incorporated herein by reference.

In one embodiment, the aerosol-generating elements 425, such as those in the form of beads or pellets, can be smoke-treated to impart smoky flavor or aroma. For example, the beads or pellets can be prepared and then subjected to smoke from a combustible source, such as a wood source (e.g., wood selected from hickory, maple, oak, apply, cherry, or mesquite). The beads or pellets can be treated with the smoke for a time sufficient to impart the desired smoky flavor or aroma, with an exemplary time range being about 5 to about 45 minutes. The manner in which the beads or pellets are contacted with smoke can vary, with one example involving heating wood chips in a container until smoke is produced (e.g., heating wood chips to a temperature of about 350-400° F.) and placing the beads or pellets to be treated within a closed environment with the smoke produced by the wood chips.

The composition of the aerosol precursor composition of the first aerosol generation arrangement and the composition of the aerosol-generating elements of the second aerosol generation arrangement are advantageously selected so as to complement one another to produce a desirable sensory experience. In certain embodiments, for example, the nicotine content of the aerosol precursor composition and the aerosol-generating elements are selected such that either or both of the aerosol precursor composition and the aerosol-generating elements may contain nicotine or a nicotinic compound or may be viewed as substantially or completely free of nicotine or a nicotinic compound. In other words, all nicotine content can be within the aerosol-generating elements or all nicotine content can be in the aerosol precursor composition or both compositions can include nicotine in some form.

In some aspects, where the aerosol-generating elements 425 comprise, for example, beads or pellets cast or extruded from materials of the various types set forth above (i.e., a graphite bead including tobacco extract and glycerin), while “damp” or otherwise before drying, may be rolled, for example, between adjacent roller elements, to flatten the shape of the respective beads/pellets. In some instances, the materials of the various types set forth above may be extruded in the form of filamentary strands, wherein the strands may be gathered to form a cylindrical rod or other suitably shaped material (i.e., relative in size to the beads/pellets used to otherwise form the aerosol generation segment) for application in the second aerosol generation arrangement 400. Upon drying, the flattened beads/pellets may then be shredded or otherwise processed to form, for example, strands, flakes, or other filler configuration that is flat or includes a planar segment that inhibits or prevents roll. Any random configurations resulting from the shredding process may be sufficient. In such instances, the flattened and shredded beads/pellets may then be included in the aerosol-generating element(s) 425, and the irregular or random configurations thereof may promote, for instance, a plurality of interstitial air spaces throughout the aerosol-generating element(s) 425, wherein the interstitial air spaces may, in turn, promote heat transfer with the individual objects within the aerosol-generating element(s) 425. That is, heating of the air in the interstitial spaces within the second aerosol generation arrangement 400 may expose more of the aerosol-generating element(s) 425 to the heat from the heating element 240, and thus result in enhanced or otherwise improved heating of the aerosol-generating element(s) 425. In other instances, the heat and the first aerosol (i.e., the combination thereof) produced by the heating element 240/atomizer 212 are directed through the porous matrix formed by the aerosol-generating element(s) 425, wherein the heated vapors passing through and heating the porous aerosol-generating element(s) 425 promotes, for example, elution (i.e., liquid, fluid, or particulate extraction; steam distillation; etc.) of an enhancement substance (i.e., a flavorant or other additive) from the aerosol-generating element(s) to the first aerosol, or otherwise promotes the enhancement substance being entrained in, imparted to, reacted with, or otherwise interacted with the first aerosol. The interaction between the enhancement substance and the first aerosol may, for example, change or alter the first aerosol, mix the enhancement substance with the first aerosol to form an enhanced aerosol or aerosol mixture, or facilitate a reaction that produces a different aerosol. In such instances, increased interstitial spaces within the aerosol-generating element(s) 425 may promote this interaction process through the second aerosol generation arrangement 400.

In some aspects, the beads/pellets may originate from a tobacco material cast on a foil/paper laminate. More particularly, the tobacco material may comprise, for example, a slurry including reconstituted tobacco, glycerin, and a binder material. Such a tobacco material is disclosed, for example, in U.S. Pat. No. 5,101,839 to Jakob et al. and U.S. Patent Application No. 2010/0186757 to Crooks et al., which are incorporated herein by reference. In addition, the slurry can incorporate granular inorganic material (i.e., calcium carbonate). The slurry is cast unto a paper element of a foil-paper laminate, such as disclosed, for example, in U.S. Pat. No. 8,678,013 to Crooks et al. and U.S. Pat. No. 7,647,932 to Cantrell et al., which is also incorporated herein by reference, and the assembled cast sheet product is then dried, for instance by the application of heat (i.e., by heated air, microwave drying, etc.). The paper element may have, for instance, a particular porosity or texture to promote an intimate contact and interaction with the slurry, for instance, over direct contact between the slurry and the foil. However, the exemplary aspect presented herein does not preclude casting the tobacco material (i.e., slurry) directly on a metal foil or other suitable thin film heat conductor. Once such a laminate is cast, the dried cast sheet (i.e., the foil/paper/tobacco material) may be shredded, diced, or otherwise separated into a plurality of cast sheet portion elements, wherein each such element preferably includes a portion of the tobacco material (i.e., the substrate) intimately interacted with a portion of the paper element which, in turn, is in intimate contact with a portion of the foil element of the foil-paper laminate. A plurality of the cast sheet portion elements may then be included in the aerosol-generating element(s) 425 forming the second aerosol generation arrangement 400.

One skilled in the art will appreciate that, in some circumstances, the cast sheet portion elements included in the aerosol-generating element(s) 425 may cooperate to promote improved heat transfer to the tobacco material forming a portion of those cast sheet portion elements or otherwise to abutting elements. More particularly, in some instances, heat transfer from the heating element 240 to the tobacco material included in the aerosol-generating element(s) 425 may be limited past any direct interface therebetween, with the heat-conducting strip forming an additional mechanism for conducting heat from the heating element 240 for heating the outer elements included in the aerosol-generating element(s) 425 and any aerosol-generating element(s) in contact therewith. In aspects including the cast sheet portion elements included in the aerosol-generating element(s) 425, the heat-conductive portions of the foil element associated with the cast sheet portion elements may form, for example, a plurality of additional heat conductive pathways. That is, the cast sheet portion elements used as all or part of the aerosol-generating element(s) 425 may provide additional heat-conductive elements interspersed throughout the aerosol-generating element(s) 425 within the second aerosol generation arrangement 400 to thereby enhance or otherwise improve heat transfer to and between the aerosol-generating elements. In achieving such an aspect, it may be further advantageous to shred or process a substrate material implemented in, for example, the cast tobacco sheet substrate material forming the substrate incorporated within the types of cigarettes commercially marketed under the trade name “Eclipse” by R. J. Reynolds Tobacco Company, as disclosed, e.g., by U.S. Pat. No. 5,469.871 to Barnes et al.

The pellets or other elements may have smooth, regular outer shapes (e.g., spheres, cylinders, ovoids, or the like) and/or they may have irregular outer shapes (e.g., shredded pieces, flakes, or the like). The aerosol-generating element(s) 425, discretely or cumulatively, may have a generally cylindrical form within the second aerosol generation arrangement 400, and may in some instances include a collection of about 800 to about 1200 generally spherical beads, each having a mean or nominal diameter of about 0.05 mm to about 4 mm (e.g., about 1 mm³ in volume, in one example), with the beads/pellets cumulatively weighing about 450 mg to about 750 mg (e.g., 600 mg±25%, in one example).

In one method of preparation, substantially spherical beads or pellets of aerosol-generating elements can be formed by first mixing together the desired composition followed by extrusion of the composition to form an extrudate. The extrudate is then processed in a spheronizer (e.g., such as spheronizers available from Caleva Process Solutions Ltd. or LCI Corporation) to produce variously-sized spheroids that can be processed through a series of screens to provide the desired size range, such as the sizes noted above.

The aerosol-generating elements can be selected so as to have relatively uniform mean diameter or a range of sizes of aerosol-generating elements can be included in the second aerosol generation arrangement 400. Where different size ranges are used in the same device, the differently sized elements can be arranged in a gradient or layers within the second aerosol generation arrangement 400 or the differently sized elements can be randomly mixed within the aerosol generation arrangement 400. Although not bound by any particular theory of operation, using aerosol-generating elements of different sizes in the same aerosol generation arrangement 400 can provide advantageous pressure drop changes in the device and/or provide advantageous sensory characteristics based on the different rates of evaporation provided by the differently sized elements.

Preferably, sufficient beads are loaded into the second aerosol generation arrangement 400 to provide at least about 95 percent of maximum fill, with beads and/or other suitable elements. It is advantageous to avoid large open pockets within the aerosol generation arrangement 400 that could allow air traveling through the aerosol generation arrangement to substantially bypass interaction with the aerosol-generating elements 425.

In some instances, a plurality of forms of the aerosol-generating element(s) 425 may be selected (e.g., aerosol-generating element(s) having different compositions) and each selected form of the aerosol-generating elements then subsequently included in the second aerosol generation arrangement 400. In other instances, the selected forms of the aerosol-generating elements may be combined, prior to inclusion in the second aerosol generation arrangement 400, to produce an aerosol-generating element mixture, and the mixture then subsequently included in the second aerosol generation arrangement 400.

The atomizer or first aerosol generation arrangement 212 and the second aerosol generation arrangement 400 may be physically separate from one another and/or comprise discrete units or segments within the cartridge body 200. In some instances, as shown, those segments may be positioned/disposed so that the downstream end (toward the mouthpiece or mouth-engaging end 220 of the cartridge body 200) of the atomizer or first aerosol generation arrangement 212 is adjacent to the upstream end of the second aerosol generation segment 400 (i.e., the back face of the aerosol-generating element(s) 425). That is, the atomizer or first aerosol generation arrangement 212 and the second aerosol generation segment 400 may be axially aligned in a serial end-to-end relationship, in some instances adjacent to or abutting one another. For example, in some instances, though physically discrete and positioned downstream from the atomizer or first aerosol generation arrangement 212, it may be desirable for the aerosol-generating element(s) 425 of the second aerosol generation arrangement 400 to physically contact the heating element 240 at the downstream end of the atomizer or first aerosol generation arrangement 212. Alternatively, those segments 212, 400 can be slightly spaced apart from one another such that the respective ends or components thereof 240, 425 are not necessarily in physical contact with the other (i.e., to prevent scorching). One skilled in the art will appreciate that, in some aspects, the second aerosol generation arrangement 400 may comprise more than one section or portion of aerosol-generating element(s) 425.

In some instances, an additional segment, spacer element, or separating element (otherwise referred to herein as “a first separating element”), acting as a spacer or screen (see, e.g., element 450 in FIG. 3) may be positioned generally perpendicular to the longitudinal axis of the cartridge body 200, wherein the first separating element 450 may provide for physical separation of those two segments 212, 400 while, in some instances, maintaining a heat conductive relationship therebetween. The first separating element 450 may, in some instances, not be conductive to heat and, in other instances, the first separating element 450 may not be electrically conductive. That is, the first separating element 450 may, but not necessarily, be heat-conductive and/or arranged to conduct heat from the heating element 240 of the atomizer/first aerosol generation arrangement 212 to the second aerosol generation arrangement 400, wherein the aerosol-generating element(s) 425 may be responsive to the heat and/or accompanying first aerosol to form a second aerosol. Further, in some instances, the first separating element 450 may be air permeable or otherwise configured to permit airflow therethrough, such that a first aerosol generated by the atomizer/first aerosol generation arrangement 400 can pass therethrough in the downstream direction. The first separating element 450 may thus also be configured and/or arranged so as to maintain the aerosol-generating element(s) 425 within the second aerosol generation arrangement 400 and separate from the atomizer/first aerosol generation arrangement 212. In still further instances, the first separating element 450 may be configured as a spacer (i.e., extending in a longitudinal direction along the cartridge body 200 so as to define a thickness) for separating the aerosol-generating element(s) 425 from the heating element 240 of the atomizer/first aerosol generation arrangement 212, for example, to minimize or prevent the aerosol-generating element(s) (i.e., beads) 425 from being scorched or burned by the heat from the heating element 240. In some instances, the first separating element 450 may also be configured as an insulator (i.e., not electrically conductive) to prevent short-circuiting of the heating element 240 in the event of contact therebetween.

Typically, the first separating element 450 is generally cylindrical or discoid in shape and of one piece construction, and is air permeable to allow the passage of drawn air through. The first separating element 450 may be heat conductive in nature, so that heat generated by the heating element 240 can be readily transported to the second aerosol generation arrangement 400. The length (thickness) of the first separating element 450 can vary, and typically extends from about less than 1 mm up to about 10 mm. In some instances, the relative longitudinal placement of the first separating element 450 within the outer body 216, spaces the interface of the first separating element 450 with the aerosol-generating element(s) 425 at between about 1 mm and up to about 20 mm (i.e., 7 mm in one example) away from the heating element 240. Typically, the first separating element 450 is comprised of a heat resistant material, such as a porous ceramic, a porous graphite material, a metal (i.e., stainless steel, brass, copper, etc.) mesh or screen, a high temperature-resistant plastic or the like. In some instances, the first separating element 450 may include, for example, longitudinally-extending air passageways formed during design/manufacture, drilled therethrough, or otherwise molded, extruded, printed (i.e., a 3D printed element using a 3D printer), or shaped into the spacer element during manufacture thereof. If desired, the first separating element 450 can incorporate catalytic materials, such as materials incorporating cerium or copper ions or oxides and/or salts of cerium and copper ions. See, for example, U.S. Pat. Nos. 8,469,035 and 8,617,263 to Banerjee et al. and U.S. Pat. Appl. Pub. No. 2007/0215168 to Banerjee et al., which are incorporated herein by reference.

In instances where the aerosol-generating element(s) 425 may be circumscribed by an insulation layer, a layer of heat conductive material (e.g., a layer or strip comprised of metal foil) may be provided therebetween (not shown). That is, representative aerosol-generating element(s) 425 include a plurality of pellets and/or other appropriate elements that can be circumscribed along its length by a layer of strip of metal foil. A representative metal foil is, for example, aluminum foil having a thickness of about 0.01 mm to about 0.05 mm. Preferably, the metal foil extends along the entire length of the outer co-axial surface of the aerosol-generating element(s) 425; and it may be preferred that the metal foil extends over (i.e., at least partially overlaps) the first separating element 450. The heat conductive material can be provided by means other than the use of metal foil. For example, the layer of metal foil can be replaced by a metal mesh or screen. Alternatively, the metal foil can be replaced by a heat conductive fabric, such as a layer or sheet of graphite fibers or heat conductive ceramic fibers. Alternatively, the heat conductive material can be provided by application of a heat conductive ink, such as a coating of ink or paint that incorporates metal particles, graphite fibers, particles of heat conductive ceramic materials, or the like.

In some aspects, another spacer element, or another separating element (otherwise referred to herein as “a second separating element”), acting as a spacer or screen (see, e.g., element 475 in FIG. 3) may be positioned generally perpendicular to the longitudinal axis of the cartridge body 200, wherein the second separating element 475 may provide for physical separation of the second aerosol generation arrangement 400 from the mouthpiece or mouth-engaging end 220 of the cartridge body 200. That is, the second separating element 475 may, but not necessarily, be heat-conductive and/or arranged to conduct heat from the second aerosol generation arrangement 400 and through the mouthpiece or mouth-engaging end 220 of the cartridge body 200. However, the second separating element 475 may be air permeable or otherwise configured to permit airflow therethrough, such that a first aerosol generated by the atomizer/first aerosol generation arrangement 212 and/or a second aerosol generated by the second aerosol generation arrangement 400, can pass therethrough in the downstream direction and through the mouthpiece or mouth-engaging end 220 of the cartridge body 200. The second separating element 475 may thus also be configured and/or arranged so as to maintain the aerosol-generating element(s) 425 within the second aerosol generation arrangement 400, without loss of any of the aerosol-generating element(s) through the mouthpiece or mouth-engaging end 220 of the cartridge body 200.

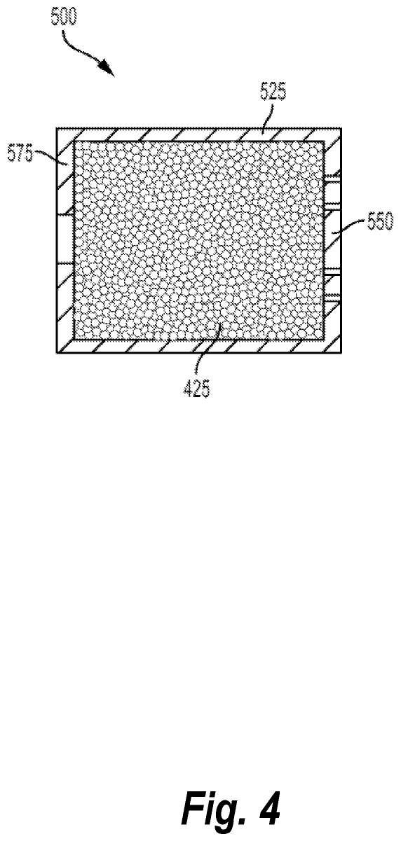

In some aspects, in the alternative to discrete first and second separating elements 450, 475 being implemented in addition to the aerosol-generating element(s) 425, the second aerosol generation arrangement 400 may comprise a cartridge 500 (see, e.g., FIG. 4) having an elongate tubular body 525 and opposed end members 550, 575, wherein each of the end members 550, 575 may be heat-conductive and/or air permeable in a similar manner to the first and second separating elements 450, 475. The elongate tubular body 525 may thus be further configured to receive the aerosol-generating element(s) 425 and to cooperate with the opposed end members 550, 575 to contain the aerosol-generating element(s) 425 therein. The assembled cartridge 500 may thus be configured to be received as a unit (forming the second aerosol generation arrangement 400) by the outer body or tubular member 216 of the cartridge body 200.

In use, the mouthpiece or mouth-engaging end 220 of the cartridge body 200 of the aerosol delivery system 100 is inserted into the mouth of the user. The atomizer/first aerosol generation arrangement 212 is then actuated, for example, by the user drawing (e.g., a suction) on the mouthpiece or mouth-engaging end 220 of the cartridge body 200. The heating element 240 and the liquid transport element 238 are configured so as to be in a heat exchange relationship. That is, the heat generated by the heating element 240 acts to heat the aerosol precursor composition carried by the liquid transport element 238 to produce a first aerosol. The heat generated by the heating element 240 and the first aerosol are then drawn into engagement with and through the second aerosol generation arrangement 400 (i.e., through the aerosol-generating element(s) 425) toward the inhalation hole defined by the mouthpiece or mouth-engaging end 220. In some instances, the heat from the heating element 240 may interact with the aerosol-generating element(s) 425 to generate a second aerosol. The second aerosol may interact or mix with the first aerosol to form a tertiary aerosol, the tertiary aerosol being the aerosol delivered to the user by way of the mouthpiece 220 in response to the draw imparted thereto by the user. In some instances, the interaction between the heat and/or the first aerosol and the aerosol-generating element(s) 425 may cause an enhancement substance to be imparted to the first aerosol so as to produce an enhanced aerosol. For example, a medicament adsorbed on the aerosol-generating element(s) 425 may react with the first aerosol and/or the heat, or otherwise be de-adsorbed from the aerosol-generating element(s) 425 by the first aerosol and/or the heat, and combine with the first aerosol to form the enhanced aerosol. In still other instances, the aerosol-generating element(s) 425 may be configured such that interaction of the first aerosol therewith causes heat to be drawn away from the first aerosol (i.e., cooling of the first aerosol). When appropriately implemented by the user, at least the first aerosol generated by the atomizer 212 and affected by the second aerosol generation arrangement 400 aerosol are generated and drawn into the mouth of the user.

The components of the second aerosol generation arrangement 400 and/or the aerosol-generating element(s) 425 therein can vary. In general, the second aerosol generation arrangement 400 and/or the aerosol-generating element(s) 425 therein may incorporate components that can be vaporized, aerosolized or entrained in air drawn through the aerosol delivery system 100 during use. Most preferably, those components, by themselves or in cooperation with the first aerosol produced by the first aerosol generation arrangement 212, provide sensory and organoleptic effects, such as aroma, flavor, mouthfeel, visible aerosol sensations, and the like. Examples of components of the first and/or second aerosol generation arrangement 212, 400 that are drawn into the mouth of the user during draw include water (e.g., as water vapor), visible or not visible aerosol forming materials (e.g., glycerin), various volatile flavors (including the plant-derived flavorants and/or synthetic flavor compound(s) disclosed herein, and optionally including one or more additional flavorants, e.g., vanillin and menthol), volatile components of tobacco (e.g., nicotine), and the like. In some embodiments comprising two aerosol generation compositions, the plant-derived flavorants and/or synthetic flavor compound(s) disclosed herein are provided in the first composition only; in some such embodiments, the plant-derived flavorants and/or synthetic flavor compound(s) disclosed herein are provided in the second composition only; and in further embodiments, the plant-derived flavorants and/or synthetic flavor compound(s) disclosed herein are provided in the first and second composition (where they may be provided, for example, in different quantities and/or where they may provide a combination of flavors/aromas by incorporating different plant-derived flavorants and/or synthetic flavor compound(s) disclosed herein).

Although an article according to the disclosure and incorporating the disclosed plant-derived flavorants and/or synthetic flavor compound(s) disclosed herein, may take on a variety of embodiments, as discussed in detail below, the use of the article by a consumer will be similar in scope. In particular, the article can be provided as a single unit or as a plurality of components that are combined by the consumer for use and then are dismantled by the consumer thereafter. Generally, a smoking article according to the disclosure can comprise a first unit that is engagable and disengagable with a second unit, the first unit comprising the resistive heating element, and the second unit comprising the electrical power source. In some embodiments, the second unit further can comprise one or more control components that actuate or regulate current flow from the electrical power source. The first unit can comprise a distal end that engages the second unit and an opposing, proximate end that includes a mouthpiece (or simply the mouthend) with an opening at a proximate end thereof. The first unit can comprise an air flow path opening into the mouthpiece of the first unit, and the air flow path can provide for passage of aerosol formed from the resistive heating element into the mouthpiece. In preferred embodiments, the first unit can be disposable. Likewise, the second unit can be reusable.

During use, the consumer initiates heating of the resistive heating element, the heat produced by the resistive heating element aerosolizes the aerosol precursor composition and, optionally, further inhalable substances. Such heating releases at least a portion of the aerosol precursor composition in the form of an aerosol (which can include any further inhalable substances included therewith), and such aerosol is provided within a space inside the cartridge that is in fluid communication with the mouthend of the cartridge. When the consumer inhales on the mouth end of the cartridge, air is drawn through the cartridge, and the combination of the drawn air and the aerosol is inhaled by the consumer as the drawn materials exit the mouth end of the cartridge (and any optional mouthpiece present) into the mouth of the consumer. To initiate heating, the consumer may actuate a pushbutton, capacitive sensor, or similar component that causes the resistive heating element to receive electrical energy from the battery or other energy source (such as a capacitor). The electrical energy may be supplied for a pre-determined length of time or may be manually controlled. Preferably, flow of electrical energy does not substantially proceed in between puffs on the article (although energy flow may proceed to maintain a baseline temperature greater than ambient temperature—e.g., a temperature that facilitates rapid heating to the active heating temperature). In further embodiments, heating may be initiated by the puffing action of the consumer through use of various sensors, as otherwise described herein. Once the puff is discontinued, heating will stop or be reduced. When the consumer has taken a sufficient number of puffs so as to have released a sufficient amount of the inhalable substance (e.g., an amount sufficient to equate to a typical smoking experience), the cartridge can be removed from the control housing and discarded. Indication that the cartridge is spent (i.e., the aerosol precursor composition has been substantially removed by the consumer) can be provided. In some embodiments, a single cartridge can provide more than a single smoking experience and thus may provide a sufficient content of aerosol precursor composition to simulate as much as a full pack of conventional cigarettes or even more.

Although the various figures described herein illustrate the control body and the cartridge in a working relationship, it is understood that the control body and the cartridge can exist as individual devices. Accordingly, any discussion otherwise provided herein in relation to the components in combination also should be understood as applying to the control body and the cartridge as individual and separate components.

In another aspect, the invention can be directed to kits that provide a variety of components as described herein. For example, a kit can comprise a control body with one or more cartridges. A kit further can comprise a control body with one or more charging components. A kit further can comprise a control body with one or more batteries. A kit further may comprise a control body with one or more cartridges and one or more charging components and/or one or more batteries. In further embodiments, a kit may comprise a plurality of cartridges. A kit further may comprise a plurality of cartridges and one or more batteries and/or one or more charging components. The inventive kits further can include a case (or other packaging, carrying, or storage component) that accommodates one or more of the further kit components. The case can be a reusable hard or soft container. Further, the case can be simply a box or other packaging structure.

In a further embodiment, the smoking article into which the disclosed flavorants is incorporated is a heat-not-burn (HNB) product or a “tobacco heating product.” Certain such products are described, for example, in U.S. patent application Ser. No. 16/110,223 to Hejazi et al., filed Aug. 23, 2018, which is incorporated by reference herein in its entirety. Further various manners and methods for incorporating tobacco into smoking articles, and particularly smoking articles that are designed so as to not purposefully burn virtually all of the tobacco within those smoking articles are set forth in U.S. Pat. No. 4,947,874 to Brooks et al.; U.S. Pat. No. 7,647,932 to Cantrell et al.; U.S. Pat. No. 8,079,371 to Robinson et al.; U.S. Pat. No. 7,290,549 to Banerjee et al.; and U.S. Pat. App. Pub. No. 2007/0215167 to Crooks et al.; the disclosures of which are incorporated herein by reference in their entireties. Some such tobacco heating products can comprise a solid or semi-solid material that may be a tobacco or tobacco-derived material. In some implementations, such a material may comprise tobacco-containing beads, tobacco shreds, tobacco strips, reconstituted tobacco material (e.g., an extruded or caste sheet substrate), or combinations thereof, and/or a mix of finely ground tobacco, tobacco extract, spray dried tobacco extract, or other tobacco form mixed with optional inorganic materials (such as calcium carbonate), optional flavors, and aerosol forming materials to form a substantially solid or moldable (e.g., extrudable) substrate. Gels and suspensions may also be utilized. Some representative types of solid and semi-solid aerosol generating component constructions and formulations are disclosed in U.S. Pat. No. 8,424,538 to Thomas et al.; U.S. Pat. No. 8,464,726 to Sebastian et al.; U.S. Pat. App. Pub. No. 2015/0083150 to Conner et al.; U.S. Pat. App. Pub. No. 2015/0157052 to Ademe et al.; and U.S. Pat. App. Pub. No. 2017/0000188 to Nordskog et al., filed Jun. 30, 2015, all of which are incorporated by reference herein. The disclosed flavorants can be incorporated within such devices with the tobacco or tobacco-derived material (e.g., as flavorant beads along with tobacco-containing beads, as flavorant mixed with the tobacco or tobacco-derived material or as a reconstituted material comprising both tobacco and flavorant) or in a portion of the device separate from the tobacco or tobacco-derived material.

In a still further embodiment, the disclosed flavorants can be incorporated within a smokeless tobacco product. Examples of smokeless tobacco products include loose moist snuff (e.g., snus); loose dry snuff; chewing tobacco; pelletized tobacco pieces; extruded or formed tobacco strips, pieces, rods, cylinders or sticks; finely divided ground powders; finely divided or milled agglomerates of powdered pieces and components; flake-like pieces; molded tobacco pieces; gums; rolls of tape-like films; readily water-dissolvable or water-dispersible films or strips; meltable compositions; lozenges; pastilles; or capsule-like materials possessing an outer shell and an inner region. Various types of smokeless tobacco products are described or referenced in US Pat. Pub. No 2012/0152265 to Dube et al., which is incorporated herein by reference. Further ingredients can be admixed with, or otherwise incorporated within, smokeless tobacco compositions including flavorants according to the present disclosure. Exemplary encapsulated additives are described, for example, in WO 2010/132444 to Atchley, which has been previously incorporated by reference herein. See also, the smokeless tobacco ingredients set forth in US Pat. Pub. Nos. 2012/0055494 to Hunt et al. and 2012/0199145 to Byrd et al., which are incorporated by reference herein.

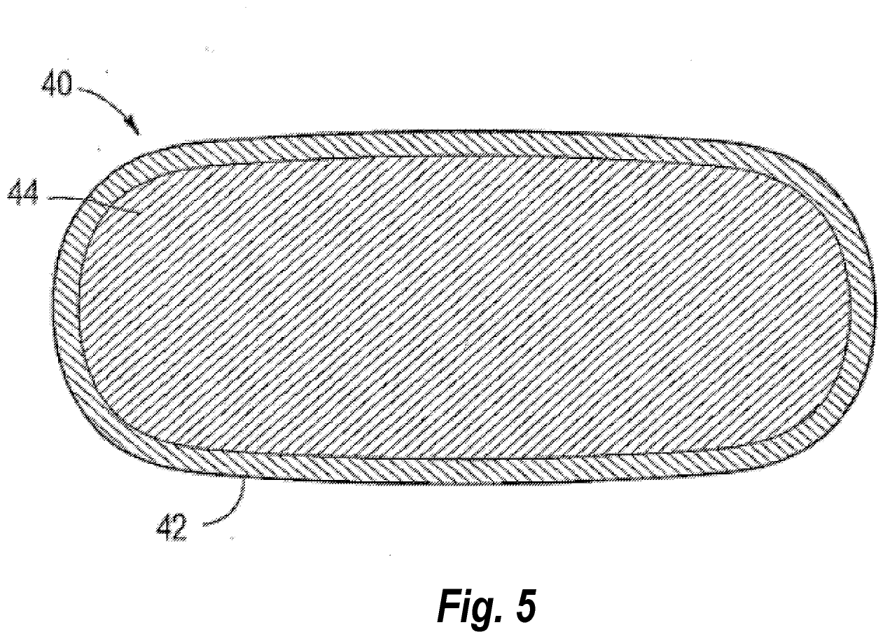

Referring to FIG. 5, a representative snus type of tobacco product comprising a concentrate or isolate of the present invention is shown. In particular, FIG. 4 illustrates a smokeless tobacco product 40 having a water-permeable outer pouch 42 containing a smokeless tobacco composition 44. Any of the components of the tobacco product can comprise a flavorant as described herein (e.g., the interior or exterior of the pouch lining or a portion of the smokeless tobacco composition contained therein).

The amount of flavorant of the present disclosure incorporated within a tobacco composition or tobacco product can depend on the desired function of the flavorant (e.g., whether it is the sole flavorant), the chemical makeup of the flavorant, and the type of tobacco composition to which the flavorant is added.

### Experimental

Aspects of the present invention are more fully illustrated by the following examples, which are set forth to illustrate certain aspects of the present invention and are not to be construed as limiting thereof.

### EXAMPLE 1

A hybrid smoking article was prepared with the following components:
1 mm Diameter Bead (containing milled hop):
   25.8% water
   15.4% Glycerin
   0.7% Carboxymethyl Cellulose (binder)
   58.1% Milled Hops
Aerosol precursor:
   60% Glycerin
   35.2% Propylene Glycol
   4.8% Nicotine package (including acids)

The beads and aerosol precursor components were formulated and incorporated within a smoking article in the manner depicted in FIG. 3 (wherein a plurality of the 1 mm diameter beads were incorporated as aerosol-generating elements 425 and the aerosol precursor was incorporated within the reservoir 214.

### EXAMPLE 2

A sample of Cascade hops (alpha acid 8.3%) was extracted in room temperature isopropanol and filtered to provide a solution. The solution was subjected to qualitative analysis using gas chromatography/mass spectrometry (GC/MS) to determine the volatile compounds associated therewith. The GC was conducted on an Agilent 7890B instrument with 30 m column (DB-WAXETR phase) at a flow rate of 1.5 mL/min under helium with 1 μL injection volume, and analyzed by MS on an Agilent 5977A instrument. Relevent parameters are noted below in Table 1.

TABLE 1
GC/MS Operating Conditions

| Parameter | Setting | | Parameter | Setting | |
|---|---|---|---|---|---|
| GC Parameters (Agilent 7890B) | | | Oven Program | | |
| Column Phase | DB-WAXETR | | Initial Temperature | 37° | C. |
| Length | 30 | m | Initial Time | 2 | min |
| Internal Diameter | 0.25 | mm | Rate 1 | 2.5° | C./min |
| Film Thickness | 0.25 | μm | Final Temperature | 230° | C. |
| Flow Mode | Constant Flow | | Final Time | 25.8 | min |
| Flow Rate | 1.5 | mL/min | Run Time | 105 | min |
| Inlet Mode | Splitless | | | | |
| Purge Flow | 50 | mL/min | | | |
| Purge Time | 0.75 | min | | | |
| Gas Saver | On | | | | |
| Gas Saver Flow | 20 | mL/min | | | |
| Gas Saver Time | 3 | min | | | |
| Gas Type | Helium | | | | |
| Inlet Temperature | 250° | C. | | | |
| Injection Volume | 1 | μL | | | |

MS Parameters (Agilent 5977A)

| Solvent Delay | 7 | min | MS acquisition mode | SCAN | |
|---|---|---|---|---|---|
| Transfer Line Temperature | 250° | C. | Mass range | 15-550 | amu |

| | | | | |
|---|---|---|---|---|
| MS Source Temperature | 230° | C. | Threshold | 150 |
| MS Quad Temperature | 150° | C. | Sampling Rate | 2 |

Peaks were correlated and assigned quality matches from the Wiley 9^{th} Library of mass spectra (indicating the degree of confidence in identification of the peak). Quality matches above 90 were considered good matches, quality matches above 80 were carefully reviewed, and quality matches below 80 were considered poor matches and were not reported. A total of 91 compounds were identified, as provided below in Table 2.

TABLE 2
Compounds Identified in Cascade hops extract using GC/MS

| RT | | | | | | | |
|---|---|---|---|---|---|---|---|
| (min) | Compound | Peak Area | | | Quality Match | | |
| 7.19 | 2,6-dimethyl-2,4-heptadiene | 1.18E+06 | 7.49E+05 | 9.07E+05 | 95 | 96 | 95 |
| 7.55 | camphene | 9.85E+04 | 1.25E+05 | 1.04E+05 | 95 | 96 | 95 |
| 7.64 | camphene | 3.83E+05 | 2.62E+05 | 3.19E+05 | 94 | 96 | 96 |
| 7.96 | isopropyl isovalerate | 3.28E+05 | 3.04E+05 | 3.13E+05 | 97 | 97 | 95 |
| 8.26 | 2,3,6-trimethyl-1,5-heptadiene | 6.67E+05 | 5.98E+05 | 6.15E+05 | 89 | 91 | 87 |
| 8.34 | isobutyl isobutyrate | 1.01E+06 | 9.62E+05 | 1.02E+06 | 93 | 94 | 93 |
| 8.53 | 2,7-dimethyl-1,6-octadiene | 3.43E+07 | 3.76E+07 | 2.76E+07 | 95 | 95 | 95 |
| 8.79 | 2,6-dimethyl-2-cis-6-octadiene | 4.79E+05 | 3.52E+05 | 3.20E+05 | 96 | 97 | 94 |
| 9.12 | 2-methylbutyl acetate | 9.76E+04 | 7.26E+04 | 6.81E+04 | 91 | 91 | 89 |
| 9.31 | carvomenthene | 1.61E+05 | 1.58E+05 | 1.39E+05 | 86 | 89 | 91 |
| 9.54 | xylene | 1.04E+05 | 8.12E+04 | 9.48E+04 | 88 | 94 | 92 |
| 10.59 | beta-myrcene | 2.61E+08 | 2.69E+08 | 2.67E+08 | 97 | 97 | 97 |
| 10.79 | alpha-terpinene | 3.21E+05 | 2.78E+05 | 2.95E+05 | 93 | 94 | 94 |
| 11.41 | limonene | 4.10E+06 | 4.02E+06 | 4.04E+06 | 98 | 98 | 98 |
| 11.59 | 2-methylbutyl isobutyrate | 6.28E+06 | 6.43E+06 | 6.20E+06 | 96 | 97 | 97 |
| 11.71 | beta-phellandrene | 2.33E+06 | 2.35E+06 | 2.28E+06 | 95 | 95 | 95 |
| 11.98 | 2-methyl-1-butanol | 1.33E+06 | 1.48E+06 | 1.33E+06 | 95 | 96 | 95 |
| 12.52 | 5,5-dimethyl-1-ethyl-1,3- | 1.72E+05 | 1.81E+05 | 2.01E+05 | 85 | 86 | 88 |
| | cyclopentadiene | | | | | | |
| 12.92 | cis-ocimene | 3.43E+05 | 2.35E+05 | 2.27E+05 | 94 | 92 | 91 |
| 13.04 | cis-ocimene | 9.41E+05 | 6.60E+05 | 5.93E+05 | 98 | 97 | 97 |
| 13.21 | tricyclene | 3.62E+05 | 2.90E+05 | 3.41E+05 | 85 | 89 | 87 |
| 13.68 | beta-ocimene | 2.06E+06 | 1.69E+06 | 1.72E+06 | 98 | 98 | 98 |
| 14.91 | 2-methylbutyl 2-methyl butyrate | 2.54E+05 | 2.96E+05 | 2.64E+05 | 89 | 92 | 90 |
| 15.63 | amyl isovalerate | 5.04E+05 | 5.36E+05 | 5.12E+05 | 97 | 96 | 95 |
| 16.78 | tiglyl alcohol | 2.71E+05 | 2.13E+05 | 1.86E+05 | 93 | 91 | 94 |
| 17.17 | 2-methylbutyl isobutyrate | 4.93E+05 | 4.99E+05 | 4.95E+05 | 93 | 92 | 94 |
| 20.29 | 1-methylene-2-methyl-3- | 4.43E+05 | 3.83E+05 | 2.42E+05 | 90 | 87 | 91 |
| | isopropenylcyclopentane | | | | | | |
| 20.94 | alpha-terpinolene | 3.41E+05 | 2.82E+05 | 2.82E+05 | 91 | 89 | 88 |
| 22.77 | acetic acid | 1.04E+06 | 1.06E+06 | 1.03E+06 | 88 | 90 | 91 |
| 23.52 | alpha-ylangene | 1.04E+06 | 9.81E+05 | 1.00E+06 | 93 | 93 | 93 |
| 23.91 | alpha-copaene | 3.62E+06 | 3.59E+06 | 3.66E+06 | 97 | 98 | 97 |
| 27.23 | 2-undecanone | 7.47E+05 | 7.23E+05 | 7.97E+05 | 83 | 83 | 82 |
| 27.47 | linalool | 3.58E+06 | 3.43E+06 | 3.51E+06 | 98 | 98 | 98 |
| 28.17 | isobutyric acid | 2.25E+06 | 2.53E+06 | 2.55E+06 | 98 | 89 | 91 |
| 28.67 | caryophyllene | 5.76E+07 | 5.93E+07 | 5.87E+07 | 99 | 99 | 99 |
| 31.96 | alpha-humulene | 1.30E+08 | 1.34E+08 | 1.32E+08 | 97 | 97 | 98 |
| 32.25 | eudesm-4-ene | 1.96E+06 | 1.98E+06 | 1.78E+06 | 96 | 96 | 96 |
| 32.68 | beta-farnesene | 6.57E+07 | 7.14E+07 | 7.05E+07 | 95 | 95 | 95 |
| 32.84 | gamma-muurolene | 6.61E+06 | 6.42E+06 | 6.37E+06 | 95 | 97 | 97 |
| 33.93 | beta-selinene | 1.11E+07 | 1.13E+07 | 1.09E+07 | 98 | 98 | 98 |
| 34.22 | alpha-selinene | 1.16E+07 | 1.11E+07 | 1.14E+07 | 97 | 96 | 97 |
| 34.47 | alpha muurolene | 3.15E+06 | 2.34E+06 | 2.73E+06 | 94 | 94 | 95 |
| 34.83 | beta-bisabolene | 1.65E+06 | 1.70E+06 | 1.57E+06 | 93 | 92 | 92 |
| 35.20 | alpha-bergamotene | 3.29E+06 | 3.40E+06 | 3.29E+06 | 94 | 94 | 93 |
| 35.41 | gamma-curcumene | 2.63E+05 | 2.79E+05 | 2.23E+05 | 87 | 87 | 89 |
| 35.81 | gamma-muurolene | 7.17E+06 | 7.63E+06 | 6.94E+06 | 96 | 98 | 97 |
| 35.88 | delta-cadinene | 1.06E+07 | 1.04E+07 | 1.08E+07 | 98 | 98 | 99 |
| 36.10 | farnesene | 2.84E+06 | 3.38E+06 | 3.27E+06 | 97 | 96 | 95 |
| 36.50 | neryl acetate | 2.57E+06 | 2.62E+06 | 2.72E+06 | 90 | 90 | 90 |
| 36.77 | 1,2,3,4,4a,7-hexahydro-1,6- | 1.52E+06 | 12352+97 | 15098+05 | 94 | 95 | 95 |
| | dimethyl-4-(1-methylethyl)- | | | | | | |
| | naphthalene | | | | | | |
| 37.22 | alpha-cadinene | 1.27E+06 | 1.28E+06 | 1.28E+06 | 96 | 96 | 97 |
| 37.49 | linalyl formate | 4.21E+05 | 4.32E+05 | 4.26E+05 | 83 | 83 | 83 |
| 38.05 | 4-methyl-2-pentenoic acid | 4.26E+05 | 5.03E+05 | 5.75E+05 | 92 | 95 | 95 |
| 38.34 | nerol | 2.33E+05 | 2.95E+05 | 2.93E+05 | 91 | 88 | 86 |
| 38.64 | 2-tridecanone | 4.20E+06 | 4.48E+06 | 4.44E+06 | 92 | 92 | 91 |
| 38.87 | neryl 2-methylpropanoate | 5.86E+06 | 6.58E+06 | 6.25E+06 | 92 | 93 | 93 |
| 39.21 | epoxycyclododecane | 2.41E+06 | 2.70E+06 | 2.32E+06 | 87 | 85 | 86 |
| 39.92 | 7-decen-2-one | 1.48E+05 | 1.58E+05 | 1.73E+05 | 82 | 82 | 80 |
| 40.21 | hexanoic acid | 3.61E+05 | 7.17E+05 | 6.19E+05 | 87 | 81 | 85 |
| 40.41 | geraniol | 1.30E+07 | 1.41E+07 | 1.38E+07 | 97 | 97 | 97 |
| 40.97 | 2-tetradecanone | 2.40E+05 | 2.50E+05 | 2.36E+05 | 84 | 83 | 84 |
| 42.67 | isoamyl acetic acid | 2.61E+05 | 3.75E+05 | 4.40E+05 | 90 | 93 | 89 |
| 43.19 | geranyl isovalerate | 3.04E+05 | 3.72E+05 | 2.29E+05 | 89 | 87 | 89 |
| 43.44 | decahydro-4H- | 2.74E+06 | 2.79E+06 | 2.96E+06 | 83 | 83 | 83 |
| | cyclopentacyclooctene | | | | | | |
| 43.71 | neophytadiene | 4.58E+06 | 3.85E+06 | 4.54E+06 | 96 | 95 | 95 |
| 44.51 | 2-methyl-2-pentenoic acid | 2.68E+07 | 3.01E+07 | 3.30E+07 | 94 | 94 | 94 |
| 44.86 | phytol | 8.55E+05 | 2.60E+05 | 3.03E+05 | 94 | 92 | 91 |
| 45.55 | e-icosyne | 3.16E+05 | 3.08E+05 | 4.03E+05 | 85 | 83 | 83 |
| 45.91 | phytol | 1.73E+06 | 1.31E+06 | 1.33E+06 | 96 | 91 | 91 |
| 46.18 | cyclododecane | 4.67E+06 | 8.46E+05 | 7.38E+05 | 86 | 82 | 80 |
| 47.11 | 2-pentadecanone | 6.98E+05 | 6.99E+05 | 6.18E+05 | 91 | 87 | 88 |
| 47.50 | cyclododecanol | 2.25E+06 | 2.40E+06 | 2.12E+06 | 85 | 85 | 86 |
| 48.15 | cubenol | 9.19E+05 | 9.76E+05 | 9.29E+05 | 90 | 86 | 86 |
| 48.66 | octanoic acid | 8.30E+05 | 9.32E+05 | 9.10E+05 | 95 | 94 | 94 |
| 49.14 | (E,Z)-1,3-cyclododecadiene | 1.48E+07 | 1.55E+07 | 1.50E+07 | 82 | 83 | 83 |
| 49.74 | geranyl hexanoate | 3.13E+05 | 1.73E+05 | 1.71E+05 | 84 | 88 | 84 |
| 51.84 | 1,5,5,8-tetramethyl-3,7- | 7.16E+05 | 6.12E+05 | 6.89E+05 | 89 | 86 | 89 |
| | cycloundecandien-1-ol | | | | | | |
| 52.27 | gamma-cadinene | 1.40E+06 | 1.44E+06 | 1.42E+06 | 89 | 89 | 89 |
| 52.59 | nonanoic acid | 1.02E+06 | 8.70E+05 | 9.19E+05 | 82 | 85 | 84 |
| 53.70 | trans-bigeranylidene | 1.39E+06 | 1.28E+06 | 1.18E+06 | 85 | 84 | 80 |
| 54.97 | juniper camphor | 1.05E+06 | 1.02E+06 | 9.85E+05 | 88 | 88 | 88 |
| 56.11 | caryophyllene oxide | 1.69E+06 | 1.77E+06 | 1.77E+06 | 89 | 88 | 86 |
| 57.83 | 9-hydroxy-linalool | 5.05E+05 | 4.83E+05 | 4.61E+05 | 88 | 84 | 83 |
| 58.13 | 9 decenoic acid | 3.62E+06 | 3.82E+06 | 3.96E+06 | 94 | 94 | 93 |
| 59.21 | farnesol isomer | 1.63E+06 | 1.78E+06 | 1.95E+06 | 92 | 91 | 90 |
| 65.81 | methyl linolenate | 2.11E+06 | 2.24E+06 | 2.40E+06 | 85 | 84 | 83 |
| 72.70 | dehydrohumulinic acid | 4.75E+06 | 5.62E+06 | 1.15E+07 | 86 | 88 | 88 |
| 73.10 | dehydrohumulinic acid | 2.46E+07 | 3.31E+07 | 4.79E+07 | 84 | 90 | 85 |
| 76.17 | palmitic acid | 1.35E+07 | 1.40E+07 | 1.52E+07 | 93 | 95 | 91 |
| 82.15 | lupulone | 9.86E+08 | 1.03E+09 | 1.06E+09 | 89 | 89 | 88 |
| 88.43 | methyl linolenate | 1.72E+07 | 1.91E+07 | 2.02E+07 | 81 | 85 | 82 |

Many modifications and other embodiments of the invention will come to mind to one skilled in the art to which this invention pertains having the benefit of the teachings presented in the foregoing description. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

What is claimed is:

1. A smoking article, comprising:
a power source;
a control component adapted to control power delivery from the power source;
a heating element; and
an aerosolizable composition comprising:
at least one flavorant derived from a plant of the humulus lupulus and/or asimina triloba species; or
at least one flavorant comprising a synthetic compound responsible for the aroma and/or flavor of an artisan food item.

2. The smoking article of claim 1, wherein the flavorant derived from a plant of the humulus lupulus species comprises milled hops.

3. The smoking article of claim 1, wherein the flavorant derived from a plant of the humulus lupulus species comprises a hop extract.

4. The smoking article of claim 3, wherein the hop extract is an extract obtained via supercritical extraction, organic solvent extraction, or hot water extraction.

5. The smoking article of claim 1, wherein the flavorant derived from a plant of the humulus lupulus species comprises a hop oil.

6. The smoking article of claim 1, wherein the flavorant derived from a plant of the humulus lupulus species comprises a flavorant derived from one or more hops selected from the group consisting of aalst, admiral, agnus, ahtanum, amarillo, aramis, azzacca, bohemie, bor, bramling cross, bravo, brewer's gold, bullion, calypso, cascade, centennial, challenger, chelan, chinook, citra, cluster, coigneau, columbus, comet, crystal, el dorado, ella, eroica, feux-coeur francais, first gold, fuggle, galaxy, galena, glacier, goldings, greenburg, green bullet, hallertau mittlefruh, hallertau herkules, hallertau magnum, hallertau taurus, harmonie, herald, hersbrucker, horizon, huell melon, junga, kohatu, liberty, Lublin, magnum, mandarina bavaria, marynka, merkur, millennium, moteuka, mount hood, mount rainier, mosaic, nelson sauvin, newport, northdown, northern brewer, nugget, opal, pacifica, pacific gem, pacific jade, kazbek, palisade, perle, phoenix, pilgrim, pilot, pioneer, polaris, polnischer lublin, poperinge, premiant, pride of ringwood, progress, rakau, riwaka, rubin, saaz, San Juan ruby red, santiam, saphir, satus, select, simcoe, sladek, smaragd, sonnet golding, sorachi ace, southern cross, spalt, sterling, sticklebract, strisselspalt, styrian atlast, styrian aurora, styrian bobek, styrian celeia, styrian golding, summer, summit, super alpha, super galena, super pride, sybilla, tardif de Bourgogne, target, tettnanger, tillicum, tomyski, topaz, tradition, ultra, vanguard, vic secret, vital, wai-iti, waimea, wakatu, whitbread golding, wilamette, zeus, and zythos hops.

7. The smoking article of claim 1, wherein the at least one flavorant comprising a synthetic compound responsible for the aroma and/or flavor of an artisan food item comprises a mixture of synthetic compounds.

8. The smoking article of claim 1, wherein the at least one flavorant comprising a synthetic compound responsible for the aroma and/or flavor of an artisan food item comprises a synthetic compound or a mixture of compounds responsible for the aroma and/or flavor of a regionally indigenous fruit.

9. The smoking article of claim 1, wherein the synthetic compound is a compound responsible for the aroma and/or flavor of a pawpaw fruit, a persimmon, a black walnut, a scuppernong grape, a muscadine grape, or an elderberry.

10. The smoking article of claim 1, wherein the at least one flavorant comprising a synthetic compound responsible for the aroma and/or flavor of an artisan food item comprises a carbonyl, an alcohol, a terpene, an aldehyde, a substituted furan, a ketone, an aromatic hydrocarbon, an aliphatic hydrocarbon, an ester, or a combination of any two or more thereof.

11. The smoking article of claim 1, wherein the at least one flavorant comprising a synthetic compound responsible for the aroma and/or flavor of an artisan food item comprises ethyl hexanoate, ethyl butanoate, methyl hexanoate, methyl octanoate, ethyl octanoate, methional, (E)-2-hexenal, phenylacetaldehyde, (E,Z)-2,6-nonadienal, hexanal, furaneol, (E,E,Z)-2,4,6-nonatrienal and (E,Z,Z)-2,4,7-decatrienal 2-pentanone, hexanal, toluene, furfural, 2-hexenal, xylene, benzaldehyde, geraniol, β-phenylethanol formate, γ-terpene, benzylalcohol, phenylethanol, linalool, nonadienal, β-ionone, butyl-2-butenoate, hexyl acetate, propyl acetate, ethyl trans-2-butenoate, hexyl-2-butenoate, ethyl acetate, butyl acetate, 1-octanol, ethyl hexanoate, β-citral, nonanal, decanal, β-citronellol, myrcenol, β-ocimene, 1-limonene, (E)-damascenone, dihydroedulan, ethyl-9-decenoate, 2-phenyl ethanol, phenylacetaldehyde, nonanal, ethyl 2-methylbutanoate, ethyl 3-methylbutanoate, methyl heptanoate, methyl octanoate, methyl nonanoate, or a combination of any two or more thereof.

12. The smoking article of claim 1, wherein the aerosolizable composition further comprises a polyhydric alcohol, a medicament, a tobacco component, a tobacco-derived material, an additional flavorant, and combinations thereof.

13. The smoking article of claim 12, wherein the polyhydric alcohol is selected from the group consisting of glycerin, propylene glycol, and combinations thereof.

14. The smoking article of claim 1, wherein the aerosolizable composition is of coated on, adsorbed by, or absorbed in at least a portion of a substrate.

15. The smoking article of claim 1, further comprising a second aerosolizable composition.

16. The smoking article of claim 15, wherein the first aerosolizable composition comprises granules, pellets, beads, discrete small units, extruded or compressed cylindrical or spherical elements, milled materials, ovoid elements, irregularly shaped elements, shredded pieces, flakes, capsules, or microcapsules comprising the at least one flavorant derived from a plant of the humulus lupulus and/or asimina triloba species; or the at least one flavorant comprising a synthetic compound responsible for the aroma and/or flavor of an artisan food item.

17. The smoking article of claim 16, wherein the first aerosolizable composition comprises beads comprising milled hops.

18. The smoking article of claim 15, comprising:
a control body portion including a first elongate tubular member having opposed ends, and a power source disposed therein;
a cartridge body portion including a second tubular member having opposed first and second ends, the first end being engaged with one of the opposed ends of the control body portion, the cartridge body portion further comprising a first aerosol generation arrangement disposed within the second tubular member and configured to operably engage the power source upon engagement between the one of the opposed ends of the control body portion and the first end of the cartridge body portion, the second end of the cartridge body portion facing toward a mouth-engaging end of the aerosol delivery system; and
a second aerosol generation arrangement disposed between the first aerosol generation arrangement and the mouth-engaging end of the aerosol delivery system, the second aerosol generation arrangement being either removably engaged with the cartridge body portion or housed within the second tubular member of the cartridge body portion,
wherein the second aerosol generation arrangement includes the first aerosolizable composition, and wherein the first aerosolizable composition comprises granules, pellets, beads, discrete small units, extruded or compressed cylindrical or spherical elements, milled materials, ovoid elements, irregularly shaped elements, shredded pieces, flakes, capsules, or microcapsules comprising the at least one flavorant derived from a plant of the humulus lupulus and/or asimina triloba species; or the at least one flavorant comprising a synthetic compound responsible for the aroma and/or flavor of an artisan food item

19. A method for providing a smoking article with flavors or aromas of craft beer, comprising:
obtaining material derived from a plant of the humulus lupulus plant; and
incorporating the material as a flavorant within the smoking article of claim 1.

20. The method of claim 19, wherein the material comprises milled hops.

21. The method of claim 19, wherein the material comprises a hop extract.

22. The method of claim 21, wherein the hop extract is an extract obtained via supercritical extraction, organic solvent extraction, or hot water extraction.

23. The method of claim 21, wherein the hop extract comprises essential oils.

24. A method for providing a smoking article with flavors or aromas of artisan foods, comprising:
identifying one or more chemical compounds associated with the flavor or aroma of a food item; and
incorporating synthetic chemical compounds identical to the one or more chemical compounds associated with the flavor of the food item as a flavorant within the smoking article of claim 1.

25. The method of claim 24, wherein the at least one flavorant comprising a synthetic compound responsible for the aroma and/or flavor of an artisan food item comprises a synthetic compound responsible for the aroma and/or flavor of a pawpaw fruit, a persimmon, a black walnut, a scuppernong grape, a muscadine grape, or an elderberry.

26. The method of claim 25, wherein the at least one flavorant comprising a synthetic compound responsible for the aroma and/or flavor of an artisan food item comprises a carbonyl, an alcohol, a terpene, an aldehyde, a substituted furan, a ketone, an aromatic hydrocarbon, an aliphatic hydrocarbon, an ester, or a combination of any two or more thereof.

27. The method of claim 25, wherein the at least one flavorant comprising a synthetic compound responsible for the aroma and/or flavor of an artisan food item comprises ethyl hexanoate, ethyl butanoate, methyl hexanoate, methyl octanoate, ethyl octanoate, methional, (E)-2-hexenal, phenylacetaldehyde, (E,Z)-2,6-nonadienal, hexanal, furaneol, (E,E,Z)-2,4,6-nonatrienal and (E,Z,Z)-2,4,7-decatrienal 2-pentanone, hexanal, toluene, furfural, 2-hexenal, xylene, benzaldehyde, geraniol, β-phenylethanol formate, γ-terpene, benzylalcohol, phenylethanol, linalool, nonadienal, β-ionone, butyl-2-butenoate, hexyl acetate, propyl acetate, ethyl trans-2-butenoate, hexyl-2-butenoate, ethyl acetate, butyl acetate, 1-octanol, ethyl hexanoate, β-citral, nonanal, decanal, β-citronellol, myrcenol, β-ocimene, 1-limonene, (E)-damascenone, dihydroedulan, ethyl-9-decenoate, 2-phenyl ethanol, phenylacetaldehyde, nonanal, ethyl 2-methylbutanoate, ethyl 3-methylbutanoate, methyl heptanoate, methyl octanoate, methyl nonanoate, or a combination of any two or more thereof.
